(19) United States
(12) Patent Application Publication
(10) Pub. No.: US 2008/0318993 A1
(43) Pub. Date: Dec. 25, 2008

(54) Method of Treating Pain Utilizing Controlled Release Oxymorphone Pharmaceutical Compositions and Instruction on Dosing for Hepatic Impairment

(75) Inventor: Harry Ahdieh, Lincoln University, PA (US)

Correspondence Address: MAYER BROWN LLP, P.O. BOX 2828, CHICAGO, IL, 60690, US

(73) Assignee: Endo Pharmaceuticals, Inc., Chadds Ford, PA (US)

(21) Appl. No.: 11/766,727

(22) Filed: Jun. 21, 2007

(51) Int. Cl. A61K 31/485; A61K 9/44; A61K 9/22

(52) U.S. Cl. 514/281; 424/10.2; 424/468; 705/3

(57) ABSTRACT

The invention pertains to a method of using oxymorphone in the treatment of pain by providing a patient with an oxymorphone dosage form and informing the patient or prescribing physician that the bioavailability of oxymorphone may be increased in patients with hepatic impairment.

### BACKGROUND OF THE INVENTION

Pain is the most frequently reported symptom and it is a common clinical problem which confronts the clinician. Many millions of people in the USA suffer from severe pain that, according to numerous recent reports, is chronically undertreated or inappropriately managed. The clinical usefulness of the analgesic properties of opioids has been recognized for centuries, and morphine and its derivatives have been widely employed for analgesia for decades in a variety of clinical pain states.

Oxymorphone HCl (14-hydroxydihydromorphinone hydrochloride) is a semi-synthetic phenanthrene-derivative opioid agonist, widely used in the treatment of acute and chronic pain, with analgesic efficacy comparable to other opioid analgesics. Oxymorphone is currently marketed as an injection (1 mg/ml in 1 ml ampules; 1.5 mg/ml in 1 ml ampules; 1.5 mg/ml in 10 ml multiple dose vials) for intramuscular, subcutaneous, and intravenous administration, and as 5 mg rectal suppositories. At one time, 2 mg, 5 mg and 10 mg oral immediate release (IR) tablet formulations of oxymorphone HCl were marketed. Oxymorphone HCl is metabolized principally in the liver and undergoes conjugation with glucuronic acid and reduction to 6-alpha- and beta-hydroxy epimers.

An important goal of analgesic therapy is to achieve continuous relief of chronic pain. Regular administration of an analgesic is generally required to ensure that the next dose is given before the effects of the previous dose have worn off. Compliance with opioids increases as the required dosing frequency decreases. Non-compliance results in suboptimal pain control and poor quality of life outcomes. (Ferrell B et al. Effects of controlled-release morphine on quality of life for cancer pain. Oncol. Nur. Forum 1989; 4:521-26). Scheduled, rather than “as needed” administration of opioids is currently recommended in guidelines for their use in chronic non-malignant pain. Unfortunately, evidence from prior clinical trials and clinical experience suggests that the short duration of action of immediate release oxymorphone would necessitate administration every 4-6 hours in order to maintain optimal levels of analgesia in chronic pain. A controlled release formulation which would allow less frequent dosing of oxymorphone would be useful in pain management.

For instance, a controlled release formulation of morphine has been demonstrated to provide patients fewer interruptions in sleep, reduced dependence on caregivers, improved compliance, enhanced quality of life outcomes, and increased control over the management of pain. In addition, the controlled release formulation of morphine was reported to provide more constant plasma concentration and clinical effects, less frequent peak to trough fluctuations, reduced dosing frequency, and possibly fewer side effects. (Thirlwell M P et al., Pharmacokinetics and clinical efficacy of oral morphine solution and controlled-release morphine tablets in cancer patients. Cancer 1989; 63:2275-83; Goughnour B R et al., Analgesic response to single and multiple doses of controlled-release morphine tablets and morphine oral solution in cancer patients. Cancer 1989; 63:2294-97; Ferrell B. et al., Effects of controlled-release morphine on quality of life for cancer pain. Oncol. Nur. Forum 1989; 4:521-26.

There are two factors associated with the metabolism of some drugs that may present problems for their use in controlled release systems. One is the ability of the drug to induce or inhibit enzyme synthesis, which may result in a fluctuating drug blood plasma level with chronic dosing. The other is a fluctuating drug blood level due to intestinal (or other tissue) metabolism or through a hepatic first-pass effect.

Oxymorphone is metabolized principally in the liver, resulting in an oral bioavailability of about 10%. Evidence from clinical experience suggests that the short duration of action of immediate release oxymorphone necessitates a four hour dosing schedule to maintain optimal levels of analgesia. It would be useful to clinicians and patients alike to have controlled release dosage forms of oxymorphone to use to treat pain and a method of treating pain using the dosage forms.

Diseases of the liver can cause impaired liver function. Examples of such diseases include hepatitis (of any type), alcoholic liver disease, toxic liver disease, and numerous others. Impaired liver function reduces the ability of the liver to process, among other things, some drugs, particularly some of those that are metabolized in the liver.

### SUMMARY OF THE INVENTION

One aspect of the invention is a method of using oxymorphone in the treatment of pain comprising providing a patient with a therapeutically effective amount of oxymorphone, and informing the patient or the patient's prescribing physician that the bioavailability of oxymorphone may be increased in patients with hepatic impairment.

Another aspect of the invention provides a method of using oxymorphone in the treatment of pain in a patient having mild hepatic impairment in need thereof comprising providing a patient having mild hepatic impairment with a therapeutically effective amount of an oral dosage form of oxymorphone, informing the patient or the patient's prescribing physician that the bioavailability of oxymorphone may be increased in patients with hepatic impairment, and orally administering the dosage form of oxymorphone to the patient.

A further aspect of the invention provides a method of using oxymorphone in the treatment of pain in a patient having mild hepatic impairment in need thereof comprising providing a patient having mild hepatic impairment with a therapeutically effective amount of a controlled release oral dosage form of oxymorphone, informing the patient or the patient's prescribing physician that the bioavailability of oxymorphone may be increased in patients with hepatic impairment, and orally administering the dosage form of oxymorphone to the patient, wherein the ratio of the log transformed plasma AUC of oxymorphone of an impaired patient to that of a healthy patient is about 0.9 to about 2.5 if both patients were administered the same dose of the composition.

### BRIEF DESCRIPTION OF THE FIGURES

FIG. 1 is a pharmacokinetic profile for 6-hydroxy oxymorphone with PID scores.

FIG. 2 is a pharmacokinetic profile for oxymorphone with PID scores.

FIG. 3 is a pharmacokinetic profile for 6-hydroxy oxymorphone with categorical pain scores.

FIG. 4 is a pharmacokinetic profile for oxymorphone with categorical pain scores.

FIG. 5 is a graph of the mean blood plasma concentration of oxymorphone versus time for clinical study 1.

FIG. 6 is a graph of the mean blood plasma concentration of oxymorphone versus time for clinical study 2.

FIG. 7 is a graph of the mean blood plasma concentration of oxymorphone versus time for clinical study 3.

FIG. 8 is a graph of the mean blood plasma concentration of 6-hydroxy oxymorphone versus time for clinical study 3.

FIG. 9 is a graph of the mean blood plasma concentration of oxymorphone for immediate and controlled release tablets from a single dose study.

FIG. 10 is a graph of the mean blood plasma concentration of oxymorphone for immediate and controlled release tablets from a steady state study.

FIG. 11 is a graph of mean plasma concentrations of oxymorphone, 6-OH-oxymorphone, and oxymorphone-3-glucuronide

FIG. 12 is a graph of the ratio and 90% confidence limits for comparison of hepatically impaired to healthy controls for oxymorphone.

FIG. 13 is a graph of the ratio and 90% confidence limits for comparison of hepatically impaired to healthy controls for 6-OH-oxymorphone.

FIG. 14 is a graph of the ratio and 90% confidence limits for comparison of hepatically impaired to healthy controls for oxymorphone-3-glucuronide.

FIG. 15 is a graph of the mean ratio (SE) of plasma metabolite AUC to oxymorphone AUC.

FIG. 16 is a graph of cumulative urinary excretion of oxymorphone and metabolites (by percent of administered dose).

FIG. 17 is a graph of the urinary excretion rate (nmol/hr) of oxymorphone and metabolites.

FIG. 18 is a graph of the relationship between oxymorphone oral clearance and measures of hepatic function.

### DETAILED DESCRIPTION OF THE INVENTION

The present invention provides methods of using oxymorphone in the treatment of pain. In one aspect of the invention the method may involve steps of providing a patient with a therapeutically effective amount of oxymorphone, and informing the patient or the patient's prescribing physician that the bioavailability of oxymorphone may be increased in patients with hepatic impairment.

Among the controlled (or extended) release, as well as immediate release, pharmaceutical compounds comprising oxymorphone that may be used in the methods of this invention is Opana®, which upon its approval on Jun. 22, 2006 became the first-ever controlled release oxymorphone formulation to be approved by the United States Food and Drug Administration (FDA). Opana® is available in both immediate release and controlled or extended release dosage forms. The approved labels of Opana® are incorporated herein by reference to the extent permitted by law.

The present invention also provides methods for alleviating pain for 12 to 24 hours using a single dose of a pharmaceutical composition by producing a blood plasma level of oxymorphone and/or 6-OH oxymorphone of at least a minimum value for at least 12 hours or more. As used herein, the terms “6-OH oxymorphone” and “6-hydroxy oxymorphone” are interchangeable and refer to the analog of oxymorphone having an alcohol (hydroxy) moiety that replaces the carboxy moiety found on oxymorphone at the 6-position.

To overcome the difficulties associated with a 4-6 hourly dosing frequency of oxymorphone, this invention provides an oxymorphone controlled release oral solid dosage form, comprising a therapeutically effective amount of oxymorphone or a pharmaceutically acceptable salt of oxymorphone. It has been found that the decreased rate of release of oxymorphone from the oral controlled release formulation of this invention does not substantially decrease the bioavailability of the drug as compared to the same dose of a solution of oxymorphone administered orally. The bioavailability is sufficiently high and the release rate is such that a sufficient plasma level of oxymorphone and/or 6-OH oxymorphone is maintained to allow the controlled release dosage to be used to treat patients suffering moderate to severe pain with once or twice daily dosing. The dosing form of the present invention can also be used with thrice daily dosing.

It is critical when considering the present invention that the difference between a controlled release tablet and an immediate release formulation be fully understood. In classical terms, an immediate release formulation releases at least 80% of its active pharmaceutical ingredient within 30 minutes. With reference to the present invention, the definition of an immediate release formulation will be broadened further to include a formulation which releases more than about 80% of its active pharmaceutical ingredient within 60 minutes in a standard USP Paddle Method dissolution test at 50 rpm in 500 ml media having a pH of between 1.2 and 6.8 at 37° C. “Controlled release” formulations, as referred to herein, will then encompass any formulations which release no more than about 80% of their active pharmaceutical ingredients within 60 minutes under the same conditions.

The controlled release dosage form of this invention exhibits a dissolution rate in vitro, when measured by USP Paddle Method at 50 rpm in 500 ml media having a pH between 1.2 and 6.8 at 37° C., of about 15% to about 50% by weight oxymorphone released after 1 hour, about 45% to about 80% by weight oxymorphone released after 4 hours, and at least about 80% by weight oxymorphone released after 10 hours.

When administered orally to humans, an effective controlled release dosage form of oxymorphone should exhibit the following in vivo characteristics: (a) peak plasma level of oxymorphone occurs within about 1 to about 8 hours after administration; (b) peak plasma level of 6-OH oxymorphone occurs within about 1 to about 8 hours after administration; (c) duration of analgesic effect is through about 8 to about 24 hours after administration; (d) relative oxymorphone bioavailability is in the range of about 0.5 to about 1.5 compared to an orally-administered aqueous solution of oxymorphone; and (e) the ratio of the area under the curve of blood plasma level vs. time for 6-OH oxymorphone compared to oxymorphone is in the range of about 0.5 to about 1.5. Of course, there is variation of these parameters among subjects, depending on the size and weight of the individual subject, the subject's age, individual metabolism differences, and other factors. Indeed, the parameters may vary in an individual from day to day. Accordingly, the parameters set forth above are intended to be mean values from a sufficiently large study so as to minimize the effect of individual variation in arriving at the values. A convenient method for arriving at such values is by conducting a study in accordance with standard FDA procedures such as those employed in producing results for use in a new drug application (or abbreviated new drug application) before the FDA. Any reference to mean values herein, in conjunction with desired results, refer to results from such a study, or some comparable study. Reference to mean values reported herein for studies actually conducted are arrived at using standard statistical methods as would be employed by one skilled in the art of pharmaceutical formulation and testing for regulatory approval.

In one specific embodiment of the controlled release matrix form of the invention, the oxymorphone or salt of oxymorphone is dispersed in a controlled release delivery system that comprises a hydrophilic material which, upon exposure to gastrointestinal fluid, forms a gel matrix that releases oxymorphone at a controlled rate. The rate of release of oxymorphone from the matrix depends on the drug's partition coefficient between components of the matrix and the aqueous phase within the gastrointestinal tract. In a preferred form of this embodiment, the hydrophilic material of the controlled release delivery system comprises a mixture of a heteropolysaccharide gum and an agent capable of cross-linking the heteropolysaccharide in presence of gastrointestinal fluid. The controlled release delivery system may also comprise a water-soluble pharmaceutical diluent mixed with the hydrophilic material. Preferably, the cross-linking agent is a homopolysaccharide gum and the inert pharmaceutical diluent is a monosaccharide, a disaccharide, or a polyhydric alcohol, or a mixture thereof.

In a specific preferred embodiment, the appropriate blood plasma levels of oxymorphone and 6-hydroxy oxymorphone are achieved using oxymorphone in the form of oxymorphone hydrochloride, wherein the weight ratio of heteropolysaccharide to homopolysaccharide is in the range of about 1:3 to about 3:1, the weight ratio of heteropolysaccharide to diluent is in the range of about 1:8 to about 8:1, and the weight ratio of heteropolysaccharide to oxymorphone hydrochloride is in the range of about 10:1 to about 1:10. A preferred heteropolysaccharide is xanthan gum and a preferred homopolysaccharide is locust bean gum. The dosage form also comprises a cationic cross-linking agent and a hydrophobic polymer. In the preferred embodiment, the dosage form is a tablet containing about 5 mg to about 80 mg of oxymorphone hydrochloride. In a most preferred embodiment, the tablet contains about 20 mg oxymorphone hydrochloride.

The invention includes a method which comprises achieving appropriate blood plasma levels of drug while providing extended pain relief by administering one to three times per day to a patient suffering moderate to severe, acute or chronic pain, an oxymorphone controlled release oral solid dosage form of the invention in an amount sufficient to alleviate the pain for a period of about 8 hours to about 24 hours. This type and intensity of pain is often associated with cancer, autoimmune diseases, infections, surgical and accidental traumas and osteoarthritis.

The invention also includes a method of making an oxymorphone controlled release oral solid dosage form of the invention which comprises mixing particles of oxymorphone or a pharmaceutically acceptable salt of oxymorphone with granules comprising the controlled release delivery system, preferably followed by directly compressing the mixture to form tablets.

Pharmaceutically acceptable salts of oxymorphone which can be used in this invention include salts with the inorganic and organic acids which are commonly used to produce nontoxic salts of medicinal agents. Illustrative examples would be those salts formed by mixing oxymorphone with hydrochloric, sulfuric, nitric, phosphoric, phosphorous, hydrobromic, maleric, malic, ascorbic, citric or tartaric, pamoic, lauric, stearic, palmitic, oleic, myristic, lauryl sulfuric, naphthylenesulfonic, linoleic or linolenic acid, and the like. The hydrochloride salt is preferred.

It has now been found that 6-OH oxymorphone, which is one of the metabolites of oxymorphone, may play a role in alleviating pain. When oxymorphone is ingested, part of the dosage gets into the bloodstream to provide pain relief, while another part is metabolized to 6-OH oxymorphone. This metabolite then enters the bloodstream to provide further pain relief. Thus it is believed that both the oxymorphone and 6-hydroxyoxymorphone levels are important to pain relief.

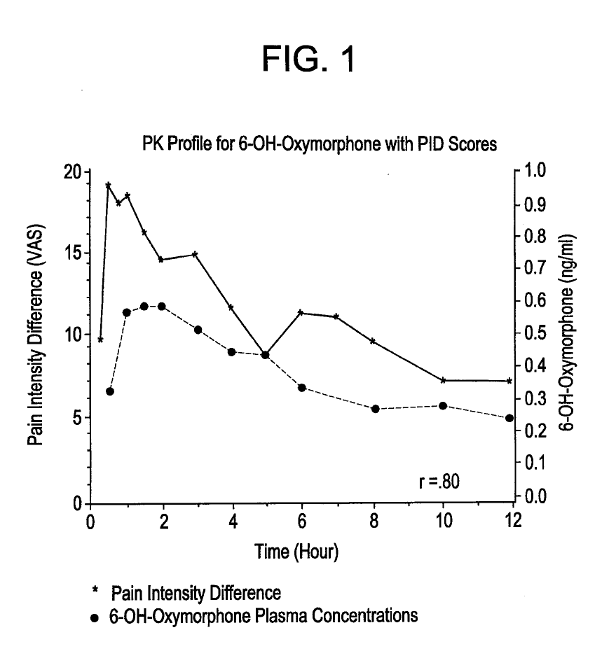

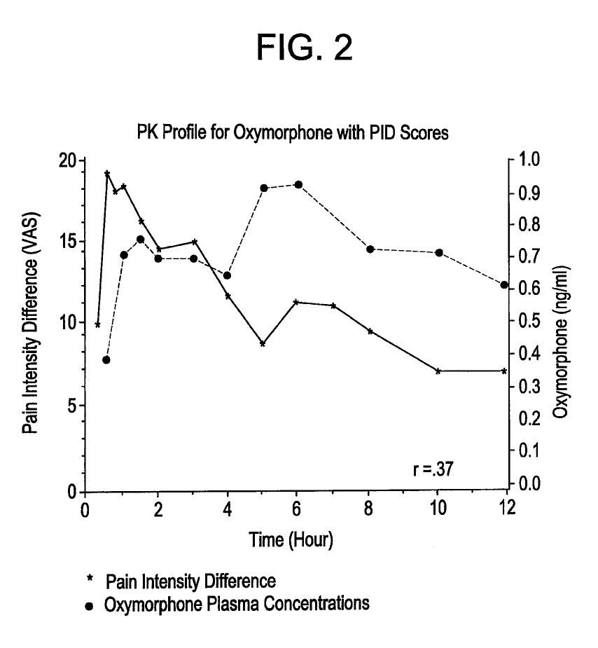

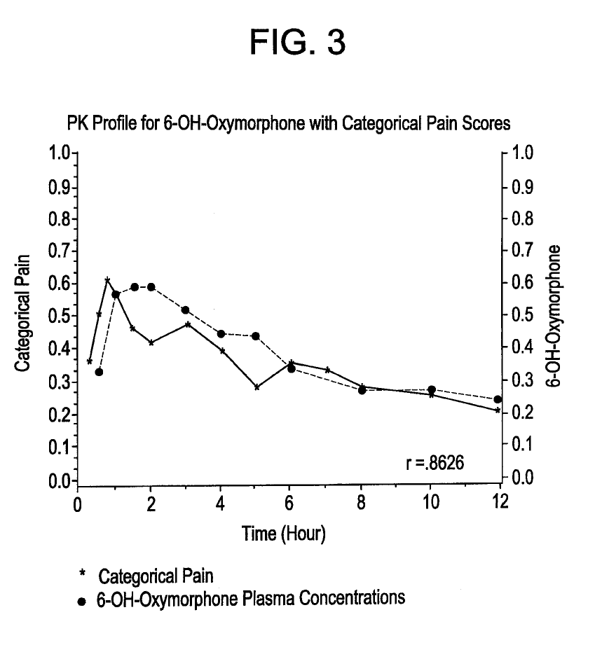

The effectiveness of oxymorphone and 6-hydroxyoxymorphone at relieving pain and the pharmacokinetics of a single dose of oxymorphone were studied. The blood plasma levels of both oxymorphone and 6-hydroxyoxymorphone were measured in patients after a single dose of oxymorphone was administered. Similarly, the pain levels in patients were measured after a single administration of oxymorphone to determine the effective duration of pain relief from a single dose. FIGS. 1-2 show the results of these tests, comparing pain levels to oxymorphone and 6-hydroxy oxymorphone levels.

For these tests, pain was measured using a Visual Analog Scale (VAS) or a Categorical Scale. The VAS scales consisted of a horizontal line, 100 mm in length. The left-hand end of the scale (0 mm) was marked with the descriptor “No Pain” and the right-hand end of the scale (100 mm) was marked with the descriptor “Extreme Pain”. Patients indicated their level of pain by making a vertical mark on the line. The VAS score was equal to the distance (in mm) from the left-hand end of the scale to the patient's mark. For the categorical scale, patients completed the following statement, “My pain at this time is” using the scale None=0, Mild=1, Moderate=2, or Severe=3.

As can be seen from these figures, there is a correlation between pain relief and both oxymorphone and 6-hydroxyoxymorphone levels. As the blood plasma levels of oxymorphone and 6-hydroxyoxymorphone increase, pain decreases (and pain intensity difference and pain relief increases). Thus, to the patient, it is the level of oxymorphone and 6-hydroxyoxymorphone in the blood plasma which is most important. Further it is these levels which dictate the efficacy of the dosage form. A dosage form which maintains a sufficiently high level of oxymorphone or 6-hydroxyoxymorphone for a longer period need not be administered frequently. Such a result is accomplished by embodiments of the present invention.

The oxymorphone controlled release oral solid dosage form of this invention can be made using any of several different techniques for producing controlled release oral solid dosage forms of opioid analgesics.

In one embodiment, a core comprising oxymorphone or oxymorphone salt is coated with a controlled release film which comprises a water insoluble material and which upon exposure to gastrointestinal fluid releases oxymorphone from the core at a controlled rate. In a second embodiment, the oxymorphone or oxymorphone salt is dispersed in a controlled release delivery system that comprises a hydrophilic material which upon exposure to gastrointestinal fluid forms a gel matrix that releases oxymorphone at a controlled rate. A third embodiment is a combination of the first two: a controlled release matrix coated with a controlled release film. In a fourth embodiment the oxymorphone is incorporated into an osmotic pump. In any of these embodiments, the dosage form can be a tablet, a plurality of granules in a capsule, or other suitable form, and can contain lubricants, colorants, diluents, and other conventional ingredients.

Osmotic Pump

An osmotic pump comprises a shell defining an interior compartment and having an outlet passing through the shell. The interior compartment contains the active pharmaceutical ingredient. Generally the active pharmaceutical ingredient is mixed with excipients or other compositions such as a polyalkylene. The shell is generally made, at least in part, from a material (such as cellulose acetate) permeable to the liquid of the environment where the pump will be used, usually stomach acid. Once ingested, the pump operates when liquid diffuses through the shell of the pump. The liquid dissolves the composition to produce a saturated situation. As more liquid diffuses into the pump, the saturated solution containing the pharmaceutical is expelled from the pump through the outlet. This produces a nearly constant release of active ingredient, in the present case, oxymorphone.

Controlled Release Coating

In this embodiment, a core comprising oxymorphone or oxymorphone salt is coated with a controlled release film which comprises a water insoluble material. The film can be applied by spraying an aqueous dispersion of the water insoluble material onto the core. Suitable water insoluble materials include alkyl celluloses, acrylic polymers, waxes (alone or in admixture with fatty alcohols), shellac and zein. The aqueous dispersions of alkyl celluloses and acrylic polymers preferably contain a plasticizer such as triethyl citrate, dibutyl phthalate, propylene glycol, and polyethylene glycol. The film coat can contain a water-soluble material such as polyvinylpyrrolidone (PVP) or hydroxypropylmethylcellulose (HPMC).

The core can be a granule made, for example, by wet granulation of mixed powders of oxymorphone or oxymorphone salt and a binding agent such as HPMC, or by coating an inert bead with oxymorphone or oxymorphone salt and a binding agent such as HPMC, or by spheronising mixed powders of oxymorphone or oxymorphone salt and a spheronising agent such as microcrystalline cellulose. The core can be a tablet made by compressing such granules or by compressing a powder comprising oxymorphone or oxymorphone salt.

The in vitro and in vivo release characteristics of this controlled release dosage form can be modified by using mixtures of different water insoluble and water soluble materials, using different plasticizers, varying the thickness of the controlled release film, including release-modifying agents in the coating, or by providing passageways through the coating.

Controlled Release Matrix

It is important in the present invention that appropriate blood plasma levels of oxymorphone and 6-hydroxy oxymorphone be achieved and maintained for sufficient time to provide pain relief to a patient for a period of 12 to 24 hours. The preferred composition for achieving and maintaining the proper blood plasma levels is a controlled-release matrix. In this embodiment, the oxymorphone or oxymorphone salt is dispersed in a controlled release delivery system that comprises a hydrophilic material (gelling agent) which upon exposure to gastrointestinal fluid forms a gel matrix that releases oxymorphone at a controlled rate. Such hydrophilic materials include gums, cellulose ethers, acrylic resins, and protein-derived materials. Suitable cellulose ethers include hydroxyalkyl celluloses and carboxyalkyl celluloses, especially hydroxyethyl cellulose (HEC), hydroxypropyl cellulose (HPC), HPMC, and carboxy methylcellulose (CMC). Suitable acrylic resins include polymers and copolymers of acrylic acid, methacrylic acid, methyl acrylate and methyl methacrylate. Suitable gums include heteropolysaccharide and homopolysaccharide gums, e.g., xanthan, tragacanth, acacia, karaya, alginates, agar, guar, hydroxypropyl guar, carrageenan, and locust bean gums.

Preferably, the controlled release tablet of the present invention is formed from (I) a hydrophilic material comprising (a) a heteropolysaccharide; or (b) a heteropolysaccharide and a cross-linking agent capable of cross-linking said heteropolysaccharide; or (c) a mixture of (a), (b) and a polysaccharide gum; and (II) an inert pharmaceutical filler comprising up to about 80% by weight of the tablet; and (III) oxymorphone.

The term “heteropolysaccharide” as used herein is defined as a water-soluble polysaccharide containing two or more kinds of sugar units, the heteropolysaccharide having a branched or helical configuration, and having excellent water-wicking properties and immense thickening properties.

A preferred heteropolysaccharide is xanthan gum, which is a high molecular weight (>10⁶) heteropolysaccharide. Other preferred heteropolysaccharides include derivatives of xanthan gum, such as deacylated xanthan gum, the carboxymethyl ether, and the propylene glycol ester.

The cross linking agents used in the controlled release embodiment of the present invention which are capable of cross-linking with the heteropolysaccharide include homopolysaccharide gums such as the galactomannans, i.e., polysaccharides which are composed solely of mannose and galactose. Galactomannans which have higher proportions of unsubstituted mannose regions have been found to achieve more interaction with the heteropolysaccharide. Locust bean gum, which has a higher ratio of mannose to the galactose, is especially preferred as compared to other galactomannans such as guar and hydroxypropyl guar.

Preferably, the ratio of heteropolysaccharide to homopolysaccharide is in the range of about 1:9 to about 9:1, preferably about 1:3 to about 3:1. Most preferably, the ratio of xanthan gum to polysaccharide material (i.e., locust bean gum, etc.) is preferably about 1:1.

In addition to the hydrophilic material, the controlled release delivery system can also contain an inert pharmaceutical diluent such as a monosaccharide, a disaccharide, a polyhydric alcohol and mixtures thereof. The ratio of diluent to hydrophilic matrix-forming material is generally in the range of about 1:3 to about 3:1.

The controlled release properties of the controlled release embodiment of the present invention may be optimized when the ratio of heteropolysaccharide gum to homopolysaccharide material is about 1:1, although heteropolysaccharide gum in an amount of from about 20 to about 80% or more by weight of the heterodisperse polysaccharide material provides an acceptable slow release product. The combination of any homopolysaccharide gums known to produce a synergistic effect when exposed to aqueous solutions may be used in accordance with the present invention. It is also possible that the type of synergism which is present with regard to the gum combination of the present invention could also occur between two homogeneous or two heteropolysaccharides. Other acceptable gelling agents which may be used in the present invention include those gelling agents well-known in the art. Examples include vegetable gums such as alginates, carrageenan, pectin, guar gum, xanthan gum, modified starch, hydroxypropylmethylcellulose, methylcellulose, and other cellulosic materials such as sodium carboxymethylcellulose and hydroxypropyl cellulose. This list is not meant to be exclusive.

The combination of xanthan gum with locust bean gum with or without the other homopolysaccharide gums is an especially preferred gelling agent. The chemistry of certain of the ingredients comprising the excipients of the present invention such as xanthan gum is such that the excipients are considered to be self-buffering agents which are substantially insensitive to the solubility of the medicament and likewise insensitive to the pH changes along the length of the gastrointestinal tract.

The inert filler of the sustained release excipient preferably comprises a pharmaceutically acceptable saccharide, including a monosaccharide, a disaccharide, or a polyhydric alcohol, and/or mixtures of any of the foregoing. Examples of suitable inert pharmaceutical fillers include sucrose, dextrose, lactose, microcrystalline cellulose, fructose, xylitol, sorbitol, mixtures thereof and the like. However, it is preferred that a soluble pharmaceutical filler such as lactose, dextrose, sucrose, or mixtures thereof be used.

The cationic cross-linking agent which is optionally used in conjunction with the controlled release embodiment of the present invention may be monovalent or multivalent metal cations. The preferred salts are the inorganic salts, including various alkali metal and/or alkaline earth metal sulfates, chlorides, borates, bromides, citrates, acetates, lactates, etc. Specific examples of suitable cationic cross-linking agents include calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate and sodium fluoride. Multivalent metal cations may also be utilized. However, the preferred cationic cross-linking agents are bivalent. Particularly preferred salts are calcium sulfate and sodium chloride. The cationic cross-linking agents of the present invention are added in an amount effective to obtain a desirable increased gel strength due to the cross-linking of the gelling agent (e.g., the heteropolysaccharide and homopolysaccharide gums). In preferred embodiments, the cationic cross-linking agent is included in the sustained release excipient of the present invention in an amount from about 1 to about 20% by weight of the sustained release excipient, and in an amount about 0.5% to about 16% by weight of the final dosage form.

In the controlled release embodiments of the present invention, the sustained release excipient comprises from about 10 to about 99% by weight of a gelling agent comprising a heteropolysaccharide gum and a homopolysaccharide gum, from about 1 to about 20% by weight of a cationic crosslinking agent, and from about 0 to about 89% by weight of an inert pharmaceutical diluent. In other embodiments, the sustained release excipient comprises from about 10 to about 75% gelling agent, from about 2 to about 15% cationic crosslinking agent, and from about 30 to about 75% inert diluent. In yet other embodiments, the sustained release excipient comprises from about 30 to about 75% gelling agent, from about 5 to about 10% cationic cross-linking agent, and from about 15 to about 65% inert diluent.

The sustained release excipient used in this embodiment of the present invention (with or without the optional cationic cross-linking agent) may be further modified by incorporation of a hydrophobic material which slows the hydration of the gums without disrupting the hydrophilic matrix. This is accomplished in preferred embodiments of the present invention by granulating the sustained release excipient with the solution or dispersion of a hydrophobic material prior to the incorporation of the medicament. The hydrophobic polymer may be selected from an alkylcellulose such as ethylcellulose, other hydrophobic cellulosic materials, polymers or copolymers derived from acrylic or methacrylic acid esters, copolymers of acrylic and methacrylic acid esters, zein, waxes, shellac, hydrogenated vegetable oils, and any other pharmaceutically acceptable hydrophobic material known to those skilled in the art. The amount of hydrophobic material incorporated into the sustained release excipient is that which is effective to slow the hydration of the gums without disrupting the hydrophilic matrix formed upon exposure to an environmental fluid. In certain preferred embodiments of the present invention, the hydrophobic material is included in the sustained release excipient in an amount from about 1 to about 20% by weight. The solvent for the hydrophobic material may be an aqueous or organic solvent, or mixtures thereof.

Examples of commercially available alkylcelluloses are Aquacoat coating (aqueous dispersion of ethylcellulose available from FMC of Philadelphia, Pa.) and Surelease coating (aqueous dispersion of ethylcellulose available from Colorcon of West Point, Pa.). Examples of commercially available acrylic polymers suitable for use as the hydrophobic material include Eudragit RS and RL polymers (copolymers of acrylic and methacrylic acid esters having a low content (e.g., 1:20 or 1:40) of quaternary ammonium compounds available from Rohm America of Piscataway, N.J.).

The controlled release matrix useful in the present invention may also contain a cationic cross-linking agent such as calcium sulfate in an amount sufficient to cross-link the gelling agent and increase the gel strength, and an inert hydrophobic material such as ethyl cellulose in an amount sufficient to slow the hydration of the hydrophilic material without disrupting it. Preferably, the controlled release delivery system is prepared as a pre-manufactured granulation.

It has now been discovered that the bioavailability of controlled-release oxymorphone is increased in patients with hepatic impairment (impaired liver function). Because of this, the oxymorphone levels in the blood of a patient with such hepatic impairment are higher than the levels that would be seen in a healthy patient receiving the same dose. As such, in order to avoid potential harmful effects, it is important to decrease the dose of controlled-release oxymorphone in patients with impaired liver function.

Since it is important that a patient or physician is aware that the bioavailability is increased so as to avoid possible issues in dosing, one embodiment of the invention comprises informing the patient or the prescribing physician that the bioavailability of oxymorphone may be increased in patients with hepatic impairment. Another embodiment of the invention comprises providing the patient or the patient's prescribing physician with prescribing information comprising instructions for dosing the controlled release oxymorphone composition to patients with hepatic impairment. For example, such instructions could be included in the labeling information, which can be for example the FDA-approved labeling, a package insert, or on the label itself. Other ways of communicating with patients or physicians are also available and are contemplated by the present invention. In another embodiment, the instructions provided comprise instructions to administer the lowest available dose. In a further embodiment, the patient or physician may be informed that the half life of oxymorphone is not significantly affected by hepatic impairment

### EXAMPLES

### Example 1

Two controlled release delivery systems are prepared by dry blending xanthan gum, locust bean gum, calcium sulfate dehydrate, and dextrose in a high speed mixed/granulator for 3 minutes. A slurry is prepared by mixing ethyl cellulose with alcohol. While running choppers/impellers, the slurry is added to the dry blended mixture, and granulated for another 3 minutes. The granulation is then dried to a LOD (loss on drying) of less than about 10% by weight. The granulation is then milled using 20 mesh screen. The relative quantities of the ingredients are listed in the table below.

TABLE 1
Cotrolled Release Delivery System

| | Formulation 1 | Formulation 2 |
|---|---|---|
| Excipient | (%) | (%) |
| Locust Bean Gum, FCC | 25.0 | 30.0 |
| Xanthan Gum, NF | 25.0 | 30.0 |
| Dextrose, USP | 35.0 | 40.0 |
| Calcium Sulfate Dihydrate, NF | 10.0 | 0.0 |
| Ethylcellulose, NF | 5.0 | 0.0 |
| Alcohol, SD3A (Anhydrous) | (10)¹ | (20.0)¹ |
| Total | 100.0 | 100.0 |

A series of tablets containing different amounts of oxymorphone hydrochloride were prepared using the controlled release delivery Formulation 1 shown in Table 1. The quantities of ingredients per tablet are as listed in the following table.

TABLE 2
Sample Tablets of Differing Strengths

| Component | Amounts in Tablet (mg) | | | | |
|---|---|---|---|---|---|
| Oxymorphone HCl, | 5 | 10 | 20 | 40 | 80 |
| USP (mg) | | | | | |
| Controlled release | 160 | 160 | 160 | 160 | 160 |
| delivery system | | | | | |
| Silicified | 20 | 20 | 20 | 20 | 20 |
| microcrystalline | | | | | |
| cellulose, N.F. | | | | | |
| Sodium stearyl | 2 | 2 | 2 | 2 | 2 |
| fumarate, NF | | | | | |
| Total weight | 187 | 192 | 202 | 222 | 262 |
| Opadry (colored) | 7.48 | 7.68 | 8.08 | 8.88 | 10.48 |
| Opadry (clear) | 0.94 | 0.96 | 1.01 | 1.11 | 1.31 |

### Examples 2, 3 and 4

Two batches of 20 mg tablets were prepared as described above, using the controlled release delivery system of Formulation 1. One batch was formulated to provide relatively fast controlled release, the other batch was formulated to provide relatively slow controlled release. Compositions of the tablets are shown in the following table.

TABLE 3
Slow and Fast Release Compositions

| | Example 2 | Example 3 | Example 4 |
|---|---|---|---|
| Ingredients | Slow (mg) | Fast (mg) | Fast (mg) |
| Oxymorphone HCl, USP | 20 | 20 | 20 |
| Controlled Release Delivery System | 360 | 160 | 160 |
| Silicified Microcrystalline Cellulose, | 20 | 20 | 20 |
| NF | | | |
| Sodium stearyl fumarate, NF | 4 | 2 | 2 |
| Total weight | 404 | 202 | 202 |
| Coating (color or clear) | 12 | 12 | 9 |

The tablets of Examples 2, 3, and 4 were tested for in vitro release rate according to USP Procedure Drug Release U.S. Pat. No. 23. Release rate is a critical variable in attempting to control the blood plasma levels of oxymorphone and 6-hydroxyoxymorphone in a patient. Results are shown in the following Table 4.

TABLE 4
Release Rates of Slow and Fast Release Tablets

| | Example 2 | Example 3 | Example 4 |
|---|---|---|---|
| Time (hr) | (Slow Release) | (Fast Release) | (Fast Release) |
| 0.5 | 18.8 | 21.3 | 20.1 |
| 1 | 27.8 | 32.3 | 31.7 |
| 2 | 40.5 | 47.4 | 46.9 |
| 3 | 50.2 | 58.5 | 57.9 |
| 4 | 58.1 | 66.9 | 66.3 |
| 5 | 64.7 | 73.5 | 74.0 |
| 6 | 70.2 | 78.6 | 83.1 |
| 8 | 79.0 | 86.0 | 92.0 |
| 10 | 85.3 | 90.6 | 95.8 |
| 12 | 89.8 | 93.4 | 97.3 |

Clinical Studies

Three clinical studies were conducted to assess the bioavailability (rate and extent of absorption) of oxymorphone. Study 1 addressed the relative rates of absorption of controlled release (CR) oxymorphone tablets (of Examples 2 and 3) and oral oxymorphone solution in fasted patients. Study 2 addressed the relative rates of absorption of CR oxymorphone tablets (of Examples 2 and 3) and oral oxymorphone solution in fed patients. Study 3 addressed the relative rates of absorption of CR oxymorphone tablets (of Example 4) and oral oxymorphone solution in fed and fasted patients.

The blood plasma levels set forth herein as appropriate to achieve the objects of the present invention are mean blood plasma levels. As an example, if the blood plasma level of oxymorphone in a patient 12 hours after administration of a tablet is said to be at least 0.5 ng/ml, any particular individual may have lower blood plasma levels after 12 hours. However, the mean minimum concentration should meet the limitation set forth. To determine mean parameters, a study should be performed with a minimum of 8 adult subjects, in a manner acceptable for filing an application for drug approval with the US Food and Drug Administration. In cases where large fluctuations are found among patients, further testing may be necessary to accurately determine mean values.

For all studies, the following procedures were followed, unless otherwise specified for a particular study.

The subjects were not to consume any alcohol-, caffeine-, or xanthine-containing foods or beverages for 24 hours prior to receiving study medication for each study period. Subjects were to be nicotine and tobacco free for at least 6 months prior to enrolling in the study. In addition, over-the-counter medications were prohibited 7 days prior to dosing and during the study. Prescription medications were not allowed 14 days prior to dosing and during the study.

Pharmacokinetic and Statistical Methods

The following pharmacokinetic parameters were computed from the plasma oxymorphone concentration-time data:

AUC₍₀₋ₜ₎ Area under the drug concentration-time curve from time zero to the time of the last quantifiable concentration (Ct), calculated using linear trapezoidal summation.

AUC_{(0-inf)} Area under the drug concentration-time curve from time zero to infinity. AUC_{(0-inf)}=AUC₍₀₋ₜ₎+Ct/Kₑₗ, where Kₑₗ is the terminal elimination rate constant.

AUC₍₀₋₂₄₎ Partial area under the drug concentration-time curve from time zero to 24 hours.

Cₘₐₓ Maximum observed drug concentration.

Tₘₐₓ Time of the observed maximum drug concentration.

Kₑₗ Elimination rate constant based on the linear regression of the terminal linear portion of the LN (concentration) time curve.

Terminal elimination rate constants for use in the above calculations were in turn computed using linear regression of a minimum of three time points, at least two of which were consecutive. Kₑₗ values for which correlation coefficients were less than or equal to 0.8 were not reported in the pharmacokinetic parameter tables or included in the statistical analysis. Thus AUC_{(0-inf)} was also not reported in these cases.

A parametric (normal-theory) general linear model was applied to each of the above parameters (excluding Tₘₐₓ), and the LN-transformed parameters Cₘₐₓ, AUC₍₀₋₂₄₎, AUC₍₀₋ₜ₎, and AUC_{(0-inf)}. Initially, the analysis of variance (ANOVA) model included the following factors: treatment, sequence, subject within sequence, period, and carryover effect. If carryover effect was not significant, it was dropped from the model. The sequence effect was tested using the subject within sequence mean square, and all other main effects were tested using the residual error (error mean square).

Plasma oxymorphone concentrations were listed by subject at each collection time and summarized using descriptive statistics. Pharmacokinetic parameters were also listed by subject and summarized using descriptive statistics.

Study 1—Two Controlled Release Formulations; Fasted Patients

Healthy volunteers received a single oral dose of 20 mg CR oxymorphone taken with 240 ml water after a 10-hour fast. Subjects received the tablets of Example 2 (Treatment 1A) or Example 3 (Treatment 1B). Further subjects were given a single oral dose of 10 mg/10 ml oxymorphone solution in 180 ml apple juice followed with 60 ml water (Treatment 1 C). The orally dosed solution was used to simulate an immediate release (IR) dose.

This study had a single-center, open-label, randomized, three-way crossover design using fifteen subjects. Subjects were in a fasted state following a 10-hour overnight fast. There was a 14-day washout interval between the three dose administrations. The subjects were confined to the clinic during each study period. Subjects receiving Treatment 1C were confined for 18 hours and subjects receiving Treatments 1A or 1B were confined for 48 hours after dosing. Ten-milliliter blood samples were collected during each study period at the 0 hour (predose), and at 0.5, 1, 1.5, 2, 3, 4, 5, 6, 7, 8, 10, 12, 14, 16, 18, 20, 24, 28, 32, 36, and 48 hours postdose for subjects receiving Treatment 1A or 1B and 0, 0.25, 0.5, 0.75, 1, 1.25, 1.5, 1.75, 2, 2.5, 3, 4, 5, 6, 7, 8, 10, 12, 14, 16, and 18 hours post-dose. The mean plasma concentration of oxymorphone versus time for each treatment across all subjects is shown in table 5.

TABLE 5
Mean Plasma Concentration vs. Time (ng/ml)

| Time (hr) | Treatment 1A | Treatment 1B | Treatment 1C |
|---|---|---|---|
| 0 | 0.000 | 0.000 | 0.0000 |
| 0.25 | | | 0.9489 |
| 0.5 | 0.2941 | 0.4104 | 1.3016 |
| 0.75 | | | 1.3264 |
| 1 | 0.5016 | 0.7334 | 1.3046 |
| 1.25 | | | 1.2041 |
| 1.5 | 0.5951 | 0.8192 | 1.0813 |
| 1.75 | | | 0.9502 |
| 2 | 0.6328 | 0.7689 | 0.9055 |
| 2.5 | | | 0.7161 |
| 3 | 0.5743 | 0.7341 | 0.6689 |
| 4 | 0.5709 | 0.6647 | 0.4879 |
| 5 | 0.7656 | 0.9089 | 0.4184 |
| 6 | 0.7149 | 0.7782 | 0.3658 |
| 7 | 0.6334 | 0.6748 | 0.3464 |
| 8 | 0.5716 | 0.5890 | 0.2610 |
| 10 | 0.4834 | 0.5144 | 0.2028 |
| 12 | 0.7333 | 0.6801 | 0.2936 |
| 14 | 0.6271 | 0.6089 | 0.2083 |
| 16 | 0.4986 | 0.4567 | 0.1661 |
| 18 | 0.4008 | 0.3674 | 0.1368 |
| 20 | 0.3405 | 0.2970 | |
| 24 | 0.2736 | 0.2270 | |
| 28 | 0.3209 | 0.2805 | |
| 32 | 0.2846 | 0.2272 | |
| 36 | 0.2583 | 0.1903 | |
| 48 | 0.0975 | 0.0792 | |

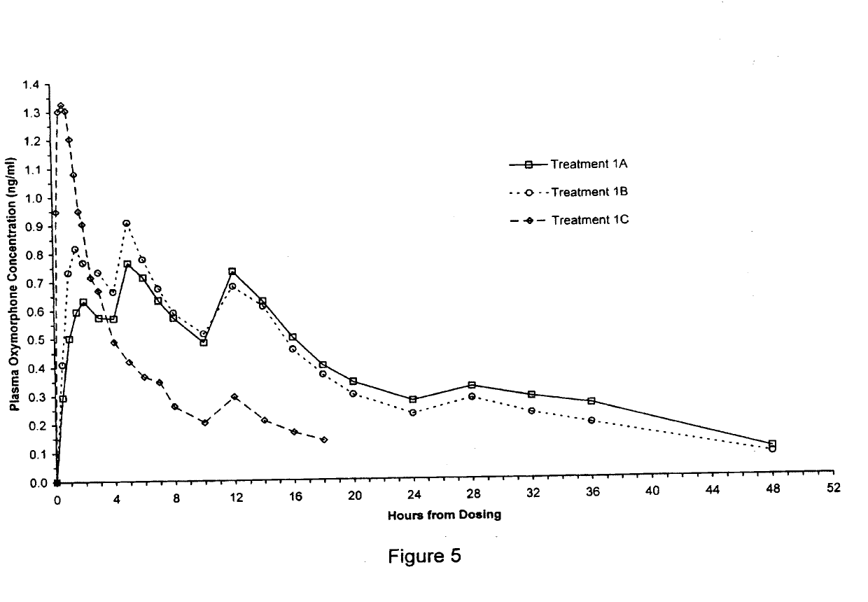

The results are shown graphically in FIG. 5. In both Table 5 and FIG. 5, the results are normalized to a 20 mg dosage. The immediate release liquid of Treatment 1C shows a classical curve, with a high and relatively narrow peak, followed by an exponential drop in plasma concentration. However, the controlled release oxymorphone tablets exhibit triple peaks in blood plasma concentration. The first peak occurs (on average) at around 3 hours. The second peak of the mean blood plasma concentration is higher than the first, occurring around 6-7 hours, on average).

Occasionally, in an individual, the first peak is higher than the second, although generally this is not the case. This makes it difficult to determine the time to maximum blood plasma concentration (Tₘₐₓ) because if the first peak is higher than the second, maximum blood plasma concentration (Cₘₐₓ) occurs much earlier (at around 3 hours) than in the usual case where the second peak is highest. Therefore, when we refer to the time to peak plasma concentration (Tₘₐₓ) unless otherwise specified, we refer to the time to the second peak. Further, when reference is made to the second peak, we refer to the time or blood plasma concentration at the point where the blood plasma concentration begins to drop the second time. Generally, where the first peak is higher than the second, the difference in the maximum blood plasma concentration at the two peaks is small. Therefore, this difference (if any) was ignored and the reported Cₘₐₓ was the true maximum blood plasma concentration and not the concentration at the second peak.

TABLE 6
Pharmacokinetic Parameters of Plasma Oxymorphone for Study 1

| | Treatment 1A | | Treatment 1B | | Treatment 1C | |
|---|---|---|---|---|---|---|
| | Mean | SD | Mean | SD | Mean | SD |
| Cₘₐₓ | 0.8956 | 0.2983 | 1.0362 | 0.3080 | 2.9622 | 1.0999 |
| Tₘₐₓ | 7.03 | 4.10 | 4.89 | 3.44 | 0.928 | 0.398 |
| AUC₍₀₋ₜ₎ | 17.87 | 6.140 | 17.16 | 6.395 | 14.24 | 5.003 |
| AUC_{(0-inf)} | 19.87 | 6.382 | 18.96 | 6.908 | 16.99 | 5.830 |
| T_{1/2el} | 10.9 | 2.68 | 11.4 | 2.88 | 6.96 | 4.61 |

Units:
Cₘₐₓ in ng/ml,
Tₘₐₓ in hours,
AUC in ng * hr/ml,
T_{1/2el} in hours.

Relative bioavailability determinations are set forth in Tables 7 and 8. For these calculations, AUC was normalized for all treatments to a 20 mg dose.

TABLE 7
Relative Bioavailability (Fᵣₑₗ) Determination Based on AUC_{(0-inf)}

| Fᵣₑₗ (1A vs. 1C) | Fᵣₑₗ (1B vs. 1C) | Fᵣₑₗ (1A vs. 1B) |
|---|---|---|
| 1.193 [symbol] 0.203 | 1.121 [symbol] 0.211 | 1.108 [symbol] 0.152 |

TABLE 8
Relative Bioavailability Determination Based on AUC₍₀₋₁₈₎

| Fᵣₑₗ (1A vs. 1C) | Fᵣₑₗ (1B vs. 1C) | Fᵣₑₗ (1A vs. 1B) |
|---|---|---|
| 0.733 [symbol] 0.098 | 0.783 [symbol] 0.117 | 0.944 [symbol] 0.110 |

Study 2—Two CR Formulations; Fed Patients

Healthy volunteers received a single oral dose of 20 mg CR oxymorphone taken with 240 ml water in a fed state. Subjects received the tablets of Example 2 (Treatment 2A) or Example 3 (Treatment 2B). Further subjects were given a single oral dose of 10 mg/10 ml oxymorphone solution in 180 ml apple juice followed with 60 ml water (Treatment 2C). The orally dosed solution was used to simulate an immediate release (IR) dose.

This study had a single-center, open-label, randomized, three-way crossover design using fifteen subjects. The subjects were in a fed state, after a 10-hour overnight fast followed by a standardized FDA high-fat breakfast. There was a 14-day washout interval between the three dose administrations. The subjects were confined to the clinic during each study period. Subjects receiving Treatment 2C were confined for 18 hours and subjects receiving Treatments 2A or 2B were confined for 48 hours after dosing. Ten-milliliter blood samples were collected during each study period at the 0 hour (predose), and at 0.5, 1, 1.5, 2, 3, 4, 5, 6, 7, 8, 10, 12, 14, 16, 18, 20, 24, 28, 32, 36, and 48 hours postdose for subjects receiving Treatment 2A or 2B and 0, 0.25, 0.5, 0.75, 1, 1.25, 1.5, 1.75, 2, 2.5, 3, 4, 5, 6, 7, 8, 10, 12, 14, 16, and 18 hours postdose. The mean plasma concentration of oxymorphone versus time for each treatment across all subjects is shown in table 9.

TABLE 9
Mean Plasma Concentration vs. Time (ng/ml)

| Time (hr) | Treatment 2A | Treatment 2B | Treatment 2C |
|---|---|---|---|
| 0 | 0.000 | 0.000 | 0.0000 |
| 0.25 | | | 1.263 |
| 0.5 | 0.396 | .0553 | 1.556 |
| 0.75 | | | 1.972 |
| 1 | 0.800 | 1.063 | 1.796 |
| 1.25 | | | 1.795 |
| 1.5 | 1.038 | 1.319 | 1.637 |
| 1.75 | | | 1.467 |
| 2 | 1.269 | 1.414 | 1.454 |
| 2.5 | | | 1.331 |
| 3 | 1.328 | 1.540 | 1.320 |
| 4 | 1.132 | 1.378 | 1.011 |
| 5 | 1.291 | 1.609 | 0.731 |
| 6 | 1.033 | 1.242 | 0.518 |
| 7 | 0.941 | 0.955 | 0.442 |
| 8 | 0.936 | 0.817 | 0.372 |
| 10 | 0.669 | 0.555 | 0.323 |
| 12 | 0.766 | 0.592 | 0.398 |
| 14 | 0.641 | 0.519 | 0.284 |
| 16 | 0.547 | 0.407 | 0.223 |
| 18 | 0.453 | 0.320 | 0.173 |
| 20 | 0.382 | 0.280 | |
| 24 | 0.315 | 0.254 | |
| 28 | 0.352 | 0.319 | |
| 32 | 0.304 | 0.237 | |
| 36 | 0.252 | 0.207 | |
| 48 | 0.104 | 0.077 | |

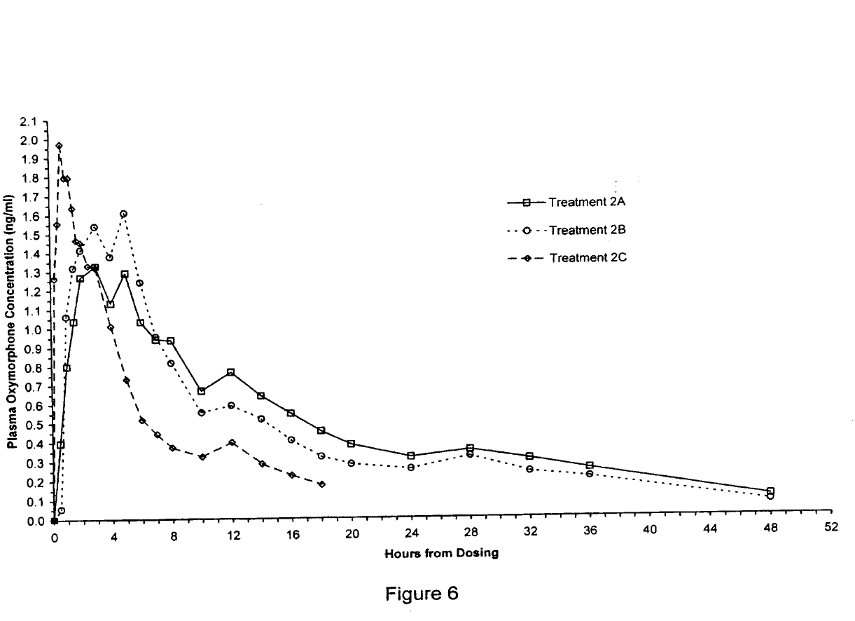

The results are shown graphically in FIG. 6. Again, the results have been normalized to a 20 mg dosage. As with Study 1, the immediate release liquid of Treatment 2C shows a classical curve, with a high and relatively narrow peak, followed by an exponential drop in plasma concentration, while the controlled release oxymorphone tablets exhibit triple peaks in blood plasma concentration. Thus, again when we refer to the time to peak plasma concentration (Tₘₐₓ) unless otherwise specified, we refer to the time to the second peak.

TABLE 10
Pharmacokinetic Parameters of Plasma Oxymorphone for Study 2

| | Treatment 2A | | Treatment 2B | | Treatment 2C | |
|---|---|---|---|---|---|---|
| | Mean | SD | Mean | SD | Mean | SD |
| Cₘₐₓ | 1.644 | 0.365 | 1.944 | 0.465 | 4.134 | 0.897 |
| Tₘₐₓ | 3.07 | 1.58 | 2.93 | 1.64 | 0.947 | 0.313 |
| AUC₍₀₋ₜ₎ | 22.89 | 5.486 | 21.34 | 5.528 | 21.93 | 5.044 |
| AUC_{(0-inf)} | 25.28 | 5.736 | 23.62 | 5.202 | 24.73 | 6.616 |
| T_{1/2el} | 12.8 | 3.87 | 11.0 | 3.51 | 5.01 | 2.02 |

Units:
Cₘₐₓ in ng/ml,
Tₘₐₓ in hours,
AUC in ng * hr/ml,
T_{1/2el} in hours.

In Table 10, the Tₘₐₓ has a large standard deviation due to the two comparable peaks in blood plasma concentration. Relative bioavailability determinations are set forth in Tables 11 and 12.

TABLE 11
Relative Bioavailability Determination Based on AUC_{(0-inf)}

| Fᵣₑₗ (2A vs. 2C) | Fᵣₑₗ (2B vs. 2C) | Fᵣₑₗ (2A vs. 2B) |
|---|---|---|
| 1.052 [symbol] 0.187 | 0.949 [symbol] 0.154 | 1.148 [symbol] 0.250 |

TABLE 12
Relative Bioavailability Determination Based on AUC₍₀₋₁₈₎

| Fᵣₑₗ (2A vs. 2C) | Fᵣₑₗ (2B vs. 2C) | Fᵣₑₗ (2A vs. 2B) |
|---|---|---|
| 0.690 [symbol] 0.105 | 0.694 [symbol] 0.124 | 1.012 [symbol] 0.175 |

As may be seen from tables 5 and 10 and FIGS. 1 and 2, the Cₘₐₓ for the CR tablets (treatments 1A, 1B, 2A and 2B) is considerably lower, and the T max much higher than for the immediate release oxymorphone. The blood plasma level of oxymorphone remains high well past the 8 (or even the 12) hour dosing interval desired for an effective controlled release tablet.

Study 3-One Controlled Release Formulation; Fed and Fasted Patients

This study had a single-center, open-label, analytically blinded, randomized, four-way crossover design. Subjects randomized to Treatment 3A and Treatment 3C, as described below, were in a fasted state following a 10-hour overnight fast. Subjects randomized to Treatment 3B and Treatment 3D, as described below, were in the fed state, having had a high fat meal, completed ten minutes prior to dosing. There was a 14-day washout interval between the four dose administrations. The subjects were confined to the clinic during each study period. Subjects assigned to receive Treatment 3A and Treatment 3B were discharged from the clinic on Day 3 following the 48-hour procedures, and subjects assigned to receive Treatment 3C and Treatment 3D were discharged from the clinic on Day 2 following the 36-hour procedures. On Day 1 of each study period the subjects received one of four treatments:

Treatments 3A and 3B: Oxymorphone controlled release 20 mg tablets from Example 3. Subjects randomized to Treatment 3A received a single oral dose of one 20 mg oxymorphone controlled release tablet taken with 240 ml of water after a 10-hour fasting period. Subjects randomized to Treatment 3B received a single oral dose of one 20 mg oxymorphone controlled release tablet taken with 240 ml of water 10 minutes after a standardized high fat meal.

Treatments 3C and 3D: oxymorphone HCl solution, USP, 1.5 mg/ml 10 ml vials. Subjects randomized to Treatment 3C received a single oral dose of 10 mg (6.7 ml) oxymorphone solution taken with 240 ml of water after a 10-hour fasting period. Subjects randomized to Treatment 3D received a single oral dose of 10 mg (6.7 ml) oxymorphone solution taken with 240 ml of water 10 minutes after a standardized high-fat meal.

A total of 28 male subjects were enrolled in the study, and 24 subjects completed the study. The mean age of the subjects was 27 years (range of 19 through 38 years), the mean height of the subjects was 69.6 inches (range of 64.0 through 75.0 inches), and the mean weight of the subjects was 169.0 pounds (range 117.0 through 202.0 pounds).

A total of 28 subjects received at least one treatment. Only subjects who completed all 4 treatments were included in the summary statistics and statistical analysis.

Blood samples (7 ml) were collected during each study period at the 0 hour (predose), and at 0.5, 1, 1.5, 2, 3, 4, 5, 6, 8, 10, 12, 14, 16, 20, 24, 30, 36, and 48 hours post-dose (19 samples) for subjects randomized to Treatment 3A and Treatment 3B. Blood samples (7 ml) were collected during each study period at the 0 hour (predose), and at 0.25, 0.5, 0.75, 1, 1.25, 1.5, 1.75, 2, 3, 4, 5, 6, 8, 10, 12, 14, 16, 20, and 36 hours post-dose (21 samples) for subjects randomized to Treatment 3C and Treatment 3D.

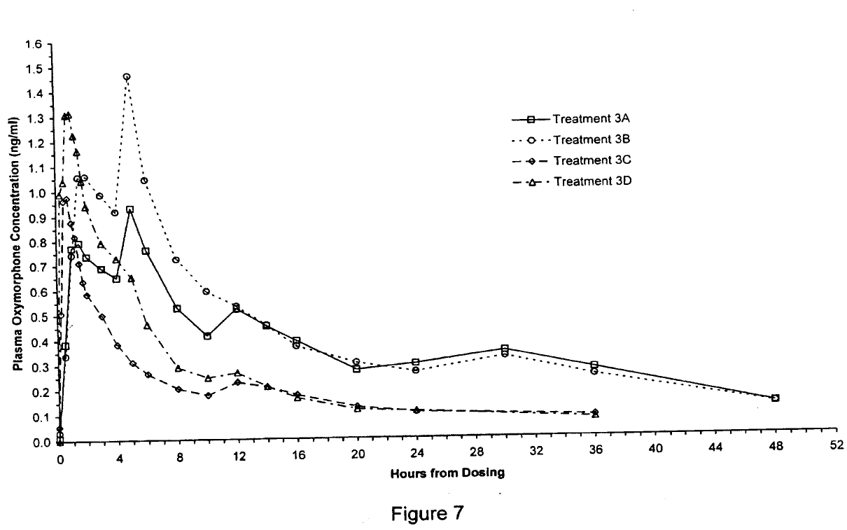

The mean oxymorphone plasma concentration versus time curves for Treatments 3A, 3B, 3C, and 3D are presented in FIG. 7. The results have been normalized to a 20 mg dosage. The data is contained in Table 13. The arithmetic means of the plasma oxymorphone pharmacokinetic parameters and the statistics for all Treatments are summarized in Table 1.

TABLE 13
Mean Plasma Concentration vs. Time (ng/ml)

| | Treatment | Treatment | Treatment | Treatment |
|---|---|---|---|---|
| Time (hr) | 3A | 3B | 3C | 3D |
| 0 | 0.0084 | 0.0309 | 0.0558 | 0.0000 |
| 0.25 | | | 0.5074 | 0.9905 |
| 0.5 | 0.3853 | 0.3380 | 0.9634 | 1.0392 |
| 0.75 | | | 0.9753 | 1.3089 |
| 1 | 0.7710 | 0.7428 | 0.8777 | 1.3150 |
| 1.25 | | | 0.8171 | 1.2274 |
| 1.5 | 1.7931 | 1.0558 | 0.7109 | 1.1638 |
| 1.75 | | | 0.6357 | 1.0428 |
| 2 | 0.7370 | 1.0591 | 0.5851 | 0.9424 |
| 3 | 0.6879 | 0.9858 | 0.4991 | 0.7924 |
| 4 | 0.6491 | 0.9171 | 0.3830 | 0.7277 |
| 5 | 0.9312 | 1.4633 | 0.3111 | 0.6512 |
| 6 | 0.7613 | 1.0441 | 0.2650 | 0.4625 |
| 8 | 0.5259 | 0.7228 | 0.2038 | 0.2895 |
| 10 | 0.4161 | 0.5934 | 0.1768 | 0.2470 |
| 12 | 0.5212 | 0.5320 | 0.2275 | 0.2660 |
| 14 | 0.4527 | 0.4562 | 0.2081 | 0.2093 |
| 16 | 0.3924 | 0.3712 | 0.1747 | 0.1623 |
| 20 | 0.2736 | 0.3021 | 0.1246 | 0.1144 |
| 24 | 0.2966 | 0.2636 | 0.1022 | 0.1065 |
| 30 | 0.3460 | 0.3231 | | |
| 36 | 0.2728 | 0.2456 | 0.0841 | 0.0743 |
| 48 | 0.1263 | 0.1241 | | |

TABLE 14
Pharmacokinetic Parameters of Plasma Oxymorphone for Study 3

| | Treatment 3A | | Treatment 3B | | Treatment 3C | | Treatment 3D | |
|---|---|---|---|---|---|---|---|---|
| | Mean | SD | Mean | SD | Mean | SD | Mean | SD |
| Cₘₐₓ | 1.7895 | 0.6531 | 1.1410 | 0.4537 | 2.2635 | 1.0008 | 2.2635 | 1.0008 |
| Tₘₐₓ | 5.65 | 9.39 | 5.57 | 7.14 | 0.978 | 1.14 | 0.978 | 1.14 |
| AUC₍ₒ₋ₜ₎ | 14.27 | 4.976 | 11.64 | 3.869 | 12.39 | 4.116 | 12.39 | 4.116 |
| AUC_{(o-inf)} | 19.89 | 6.408 | 17.71 | 8.471 | 14.53 | 4.909 | 14.53 | 4.909 |
| T_{1/2el} | 21.29 | 6.559 | 19.29 | 5.028 | 18.70 | 6.618 | 18.70 | 6.618 |
| | 12.0 | 3.64 | 12.3 | 3.99 | 16.2 | 11.4 | 16.2 | 11.4 |

The relative bioavailability calculations are summarized in tables 15 and 16.

TABLE 15
Relative Bioavailability Determination Based on AUC_{(0-inf)}

| | | Fᵣₑₗ | Fᵣₑₗ |
|---|---|---|---|
| Fᵣₑₗ (3A vs. 3C) | Fᵣₑₗ (3B vs. 3D) | (3D vs. 3C) | (3A vs. 3B) |
| 1.040 [symbol] 0.1874 | 0.8863 [symbol] 0.2569 | 1.368 [symbol] | 1.169 [symbol] |
| | | 0.4328 | 0.2041 |

TABLE 16
Relative bioavailability Determination Based on AUC₍₀₋₂₄₎

| Fᵣₑₗ (3A vs. 2C) | Fᵣₑₗ (3B vs. 3D) | Fᵣₑₗ (3D vs. 3C) | Fᵣₑₗ (3A vs. 3B) |
|---|---|---|---|
| 0.9598 [symbol] 0.2151 | 0.8344 [symbol] 0.100 | 1.470 [symbol] 0.3922 | 1.299 [symbol] 0.4638 |

The objectives of this study were to assess the relative bioavailability of oxymorphone from oxymorphone controlled release (20 mg) compared to oxymorphone oral solution (10 mg) under both fasted and fed conditions, and to determine the effect of food on the bioavailability of oxymorphone from the controlled release formulation, oxymorphone CR, and from the oral solution.

The presence of a high fat meal had a substantial effect on the oxymorphone Cₘₐₓ, but less of an effect on oxymorphone AUC from oxymorphone controlled release tablets. Least Squares (LS) mean Cₘₐₓ was 58% higher and LS mean AUC₍₀₋ₜ₎ and AUC_{(0-inf)} were 18% higher for the fed condition (Treatment B) compared to the fasted condition (Treatment A) based on LN-transformed data. This was consistent with the relative bioavailability determination from AUC_{(0-inf)} since mean Fᵣₑₗ was 1.17. Mean Tₘₐₓ values were similar (approximately 5.6 hours), and no significant difference in Tₘₐₓ was shown using nonparametric analysis. Half value durations were significantly different between the two treatments.

The effect of food on oxymorphone bioavailability from the oral solution was more pronounced, particularly in terms of AUC. LS mean Cₘₐₓ was 50% higher and LS mean AUC₍₀₋ₜ₎ and AUC_{(0-inf)} were 32-34% higher for the fed condition (Treatment D) compared to the fasted condition (Treatment C) based on LN-transformed data. This was consistent with the relative bioavailability determination from AUC_{(0-inf)} since mean Fᵣₑₗ was 1.37. Mean Tₘₐₓ (approximately 1 hour) was similar for the two treatments and no significant difference was shown.

Under fasted conditions, oxymorphone controlled release 20 mg tablets exhibited similar extent of oxymorphone availability compared to 10 mg oxymorphone oral solution normalized to a 20 mg dose (Treatment A versus Treatment C). From LN-transformed data, LS mean AUC₍₀₋ₜ₎ was 17% higher for oxymorphone CR, whereas LS mean AUC_{(0-inf)} values were nearly equal (mean ratio=99%). Mean Fᵣₑₗ values calculated from AUC_{(0-inf)} and AUC₍₀₋₂₄₎, (1.0 and 0.96, respectively) also showed similar extent of oxymorphone availability between the two treatments.

As expected, there were differences in parameters reflecting rate of absorption. LS mean Cₘₐₓ was 49% lower for oxymorphone controlled release tablets compared to the dose-normalized oral solution, based on LN-transformed data. Half-value duration was significantly longer for the controlled release formulation (means, 12 hours versus 2.5 hours).

Under fed conditions, oxymorphone availability from oxymorphone controlled release 20 mg was similar compared to 10 mg oxymorphone oral solution normalized to a 20 mg dose (Treatment B versus Treatment D). From LN-transformed data, LS mean AUC_{(0-inf)} was 12% lower for oxymorphone CR. Mean Fᵣₑₗ values calculated from AUC_{(0-inf)} and AUC₍₀₋₂₄₎, (0.89 and 0.83 respectively) also showed similar extent of oxymorphone availability from the tablet. As expected, there were differences in parameters reflecting rate of absorption. LS mean Cₘₐₓ was 46% lower for oxymorphone controlled release tablets compared to the dose-normalized oral solution, based on LN-transformed data. Mean Tₘₐₓ was 5.7 hours for the tablet compared to 1.1 hours for the oral solution. Half-value duration was significantly longer for the controlled release formulation (means, 7.8 hours versus 3.1 hours).

The presence of a high fat meal did not appear to substantially affect the availability following administration of oxymorphone controlled release tablets. LS mean ratios were 97% for AUC₍₀₋ₜ₎ and 91% for Cₘₐₓ (Treatment B versus A), based on LN-transformed data. This was consistent with the relative bioavailability determination from AUC₍₀₋₂₄₎, since mean Fᵣₑₗ was 0.97. Mean Tₘₐₓ was later for the fed treatment compared to the fasted treatment (5.2 and 3.6 hours, respectively), and difference was significant.

Under fasted conditions, oxymorphone controlled release 20 mg tablets exhibited similar availability compared to 10 mg oxymorphone oral solution normalized to a 20 mg dose (Treatment A versus Treatment C). From LN-transformed data, LS mean ratio for AUC₍₀₋ₜ₎ was 104.5%. Mean Fᵣₑₗ (0.83) calculated from AUC₍₀₋₂₄₎ also showed similar extent of oxymorphone availability between the two treatments. Mean Tₘₐₓ was 3.6 hours for the tablet compared to 0.88 for the oral solution. Half-value duration was significantly longer for the controlled release formulation (means, 11 hours versus 2.2 hours).

Under fed conditions, availability from oxymorphone controlled release 20 mg was similar compared to 10 mg oxymorphone oral solution normalized to a 20 mg dose (Treatment B versus Treatment D). From LN-transformed data, LS mean AUC₍₀₋ₜ₎ was 14% higher for oxymorphone CR. Mean Fᵣₑₗ (0.87) calculated from AUC₍₀₋₂₄₎ also indicated similar extent of availability between the treatments. Mean Tₘₐₓ was 5.2 hours for the tablet compared to 1.3 hour for the oral solution. Half-value duration was significantly longer for the controlled release formulation (means, 14 hours versus 3.9 hours).

The extent of oxymorphone availability from oxymorphone controlled release 20 mg tablets was similar under fed and fasted conditions since there was less than a 20% difference in LS mean AUC₍₀₋ₜ₎ and AUC_{(0-inf)} values for each treatment, based on LN-transformed data. Tₘₐₓ was unaffected by food; however, LS mean Cₘₐₓ was increased 58% in the presence of the high fat meal. Both rate and extent of oxymorphone absorption from the oxymorphone oral solution were affected by food since LS mean Cₘₐₓ and AUC values were increased approximately 50 and 30%, respectively. Tₘₐₓ was unaffected by food. Under both fed and fasted conditions, oxymorphone controlled release tablets exhibited similar extent of oxymorphone availability compared to oxymorphone oral solution since there was less than a 20% difference in LS mean AUC(0-t) and AUC(0-inf) values for each treatment.

Bioavailability following oxymorphone controlled release 20 mg tablets was also similar under fed and fasted conditions since there was less than a 20% difference in LS mean Cₘₐₓ and AUC values for each treatment. Tₘₐₓ was later for the fed condition. The presence of food did not affect the extent of availability from oxymorphone oral solution since LS mean AUC values were less than 20% different. However, Cₘₐₓ was decreased 35% in the presence of food. Tₘₐₓ was unaffected by food. Under both fed and fasted conditions, oxymorphone controlled release tablets exhibited similar extent of availability compared to oxymorphone oral solution since there was less than a 20% difference in LS mean AUC values for each treatment.

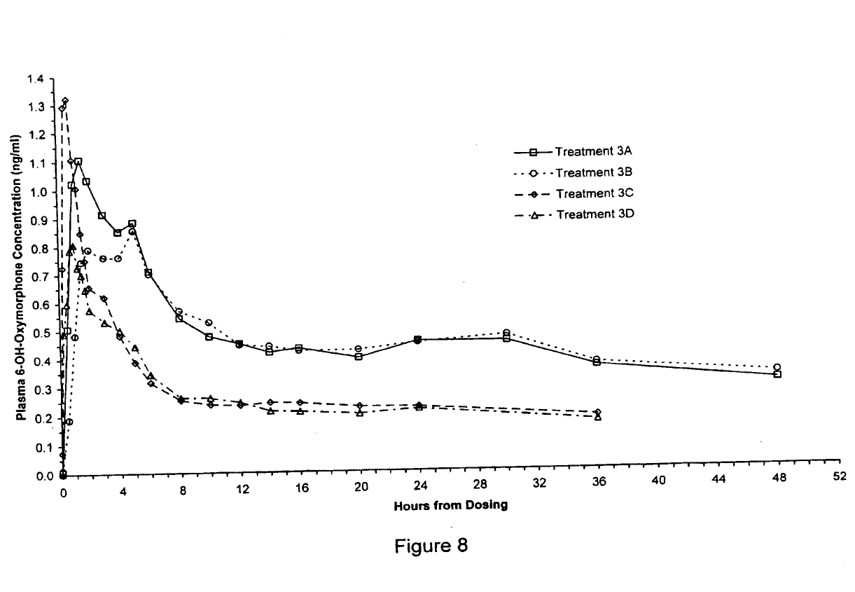

The mean 6-OH oxymorphone plasma concentration versus time curves for Treatments 3A, 3B, 3C, and 3D are presented in FIG. 8. The data is contained in Table 17.

TABLE 17
Mean Plasma Concentration vs. Time (ng/ml)

6-Hydroxyoxymorphone

| | Treatment | Treatment | Treatment | Treatment |
|---|---|---|---|---|
| Time (hr) | 3A | 3B | 3C | 3D |
| 0 | 0.0069 | 0.0125 | 0.0741 | 0.0000 |
| 0.25 | | | 0.7258 | 0.4918 |
| 0.5 | 0.5080 | 0.1879 | 1.2933 | 0.5972 |
| 0.75 | | | 1.3217 | 0.7877 |
| 1 | 1.0233 | 0.4830 | 1.1072 | 0.8080 |
| 1.25 | | | 1.0069 | 0.7266 |
| 1.5 | 1.1062 | 0.7456 | 0.8494 | 0.7001 |
| 1.75 | | | 0.7511 | 0.6472 |
| 2 | 1.0351 | 0.7898 | 0.6554 | 0.5758 |
| 3 | 0.9143 | 0.7619 | 0.6196 | 0.5319 |
| 4 | 0.8522 | 0.7607 | 0.4822 | 0.5013 |
| 5 | 0.8848 | 0.8548 | 0.3875 | 0.4448 |
| 6 | 0.7101 | 0.7006 | 0.3160 | 0.3451 |
| 8 | 0.5421 | 0.5681 | 0.2525 | 0.2616 |
| 10 | 0.4770 | 0.5262 | 0.2361 | 0.2600 |
| 12 | 0.4509 | 0.4454 | 0.2329 | 0.2431 |
| 14 | 0.4190 | 0.4399 | 0.2411 | 0.2113 |
| 16 | 0.4321 | 0.4230 | 0.2385 | 0.2086 |
| 20 | 0.3956 | 0.4240 | 0.2234 | 0.1984 |
| 24 | 0.4526 | 0.4482 | 0.2210 | 0.2135 |
| 30 | 0.4499 | 0.4708 | | |
| 36 | 0.3587 | 0.3697 | 0.1834 | 0.1672 |
| 48 | 0.3023 | 0.3279 | | |

TABLE 18
Pharmacokinetic Parameters of Plasma Oxymorphone for Study 3

| | Treatment 3A | | Treatment 3B | | Treatment 3C | | Treatment 3D | |
|---|---|---|---|---|---|---|---|---|
| | Mean | SD | Mean | SD | Mean | SD | Mean | SD |
| Cₘₐₓ | 1.2687 | 0.5792 | 1.1559 | 0.4848 | 1.5139 | 0.7616 | 0.9748 | 0.5160 |
| Tₘₐₓ | 3.61 | 7.17 | 5.20 | 9.52 | 0.880 | 0.738 | 1.30 | 1.04 |
| AUC₍ₒ₋ₜ₎ | 22.47 | 10.16 | 22.01 | 10.77 | 10.52 | 4.117 | 9.550 | 4.281 |
| AUC_{(o-inf)} | 38.39 | 23.02 | 42.37 | 31.57 | 20.50 | 7.988 | 23.84 | 11.37 |
| T_{1/2el} | 39.1 | 36.9 | 39.8 | 32.6 | 29.3 | 12.0 | 44.0 | 35.00 |

Study 4-Controlled Release 20 mg vs Immediate Release 10 mg

A study was conducted to compare the bioavailability and pharmacokinetics of controlled release and immediate release oxymorphone tablets under single-dose and multiple-dose (steady state) conditions. For the controlled release study, healthy volunteers received a single dose of a 20 mg controlled release oxymorphone table on the morning of Day 1. Beginning on the morning of Day 3, the volunteers were administered a 20 mg controlled release oxymorphone tablet every 12 hours through the morning dose of Day 9. For the immediate release study, healthy volunteers received a single 10 mg dose of an immediate release oxymorphone tablet on the morning of Day 1. On the morning of Day 3, additional 10 mg immediate release tablets were administered every six hours through the first two doses on Day 9.

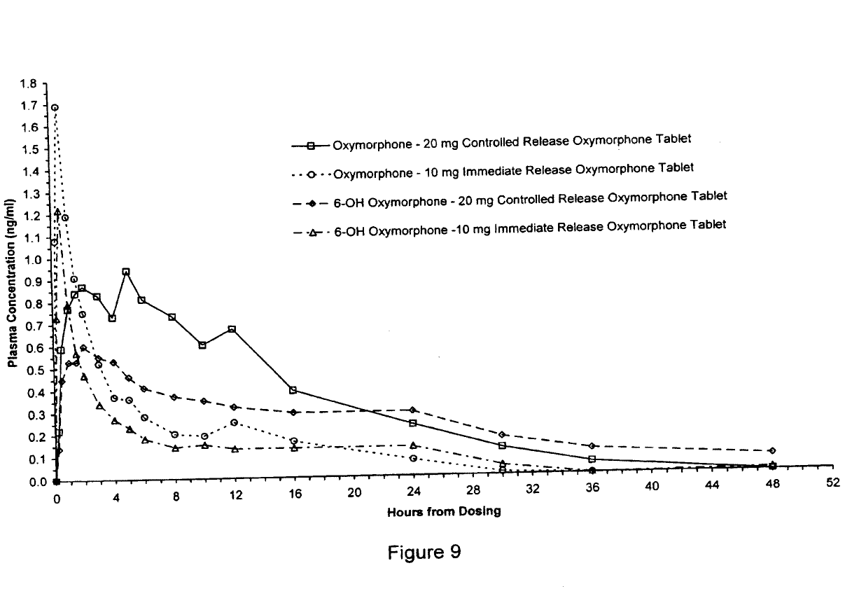

FIG. 9 shows the average plasma concentrations of oxymorphone and 6-6-hydroxy oxymorphone for all subjects after a single dose either controlled release (CR) 20 mg or immediate release (IR) 10 mg oxymorphone. The data in the figure (as with the other relative experimental data herein) is normalized to a 20 mg dose. The immediate release tablet shows a classical curve, with a high, relatively narrow peak followed by an exponential drop in plasma concentration. The controlled release oxymorphone tablets show a lower peak with extended moderate levels of oxymorphone and 6-hydroxy oxymorphone. Table 19 shows the levels of oxymorphone and 6-hydroxy oxymorphone from FIG. 9 in tabular form.

TABLE 19
Mean Plasma Concentration (ng/ml)

| | |
|---|---|
| | 6- |
| Oxymorphone | Hydroxyoxymorphone |

| | Controlled | Immediate | Controlled | Immediate |
|---|---|---|---|---|
| | Release | Release | Release | Release |
| Hour | 20 mg | 10 mg | 20 mg | 10 mg |
| 0.00 | 0.00 | 0.00 | 0.00 | 0.00 |
| 0.25 | 0.22 | 1.08 | 0.14 | 0.73 |
| 0.50 | 0.59 | 1.69 | 0.45 | 1.22 |
| 1.00 | 0.77 | 1.19 | 0.53 | 0.79 |
| 1.50 | 0.84 | 0.91 | 0.53 | 0.57 |
| 2.00 | 0.87 | 0.75 | 0.60 | 0.47 |
| 3.00 | 0.83 | 0.52 | 0.55 | 0.34 |
| 4.00 | 0.73 | 0.37 | 0.53 | 0.27 |
| 5.00 | 0.94 | 0.36 | 0.46 | 0.23 |
| 6.00 | 0.81 | 0.28 | 0.41 | 0.18 |
| 8.00 | 0.73 | 0.20 | 0.37 | 0.14 |
| 10.0 | 0.60 | 0.19 | 0.35 | 0.15 |
| 12.0 | 0.67 | 0.25 | 0.32 | 0.13 |
| 16.0 | 0.39 | 0.16 | 0.29 | 0.13 |
| 24.0 | 0.23 | 0.07 | 0.29 | 0.13 |
| 30.0 | 0.12 | 0.01 | 0.17 | 0.04 |
| 36.0 | 0.05 | 0.00 | 0.11 | 0.00 |
| 48.0 | 0.00 | 0.00 | 0.07 | 0.01 |

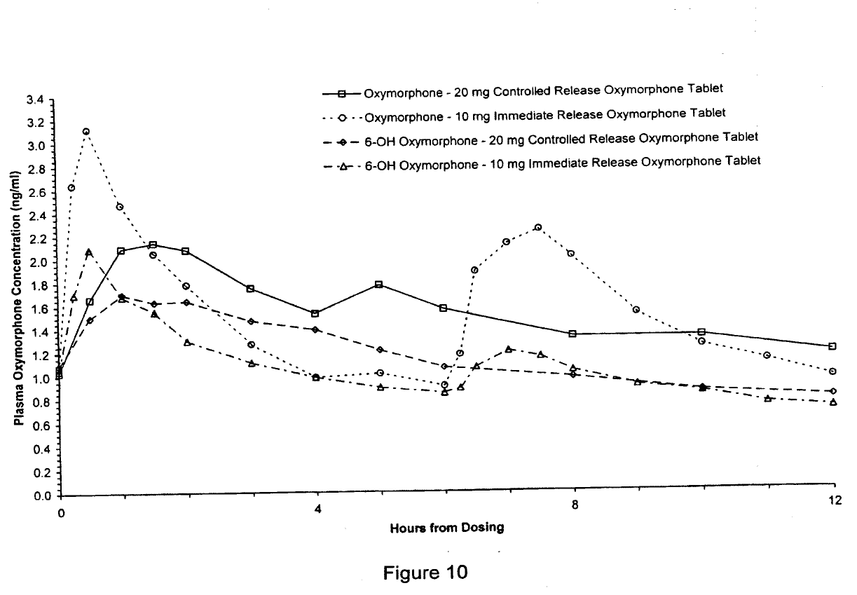

FIG. 10 shows the average plasma concentrations of oxymorphone and 6-hydroxyoxymorphone for all subjects in the steady state test, for doses of controlled release 20 mg tablets and immediate release 10 mg tablets of oxymorphone. The figure shows the plasma concentrations after the final controlled release tablet is given on Day 9, and the final immediate release tablet is given 12 hours thereafter. The steady state administration of the controlled release tablets clearly shows a steady moderate level of oxymorphone ranging from just over 1 ng/ml to almost 1.75 ng/ml over the course of a twelve hour period, where the immediate release tablet shows wide variations in blood plasma concentration. Table 20 shows the levels of oxymorphone and 6-hydroxyoxymorphone from FIG. 10 in tabular form.

TABLE 20
Summary of Mean Plasma Concentration (ng/ml)

| | | Oxymorphone | | 6-Hydroxyoxymorphone | |
|---|---|---|---|---|---|
| | | Controlled | Immediate | Controlled | Immediate |
| | | Release | Release | Release | Release |
| Day | Hour | 20 mg | 10 mg | 20 mg | 10 mg |
| 4 | 0.00 | 1.10 | 0.75 | 0.89 | 0.72 |
| 5 | 0.00 | 1.12 | 0.84 | 1.15 | 0.88 |
| 6 | 0.00 | 1.20 | 0.92 | 1.15 | 0.87 |
| 7 | 0.00 | 1.19 | 0.91 | 1.27 | 1.00 |
| 8 | 0.00 | 1.19 | 0.86 | 1.29 | 0.98 |
| 9 | 0.00 | 1.03 | 1.07 | 1.09 | 1.05 |
| | 0.25 | | 2.64 | | 1.70 |
| | 0.50 | | 3.12 | 1.50 | 2.09 |
| | 1.00 | | 2.47 | 1.70 | 1.68 |
| | 1.50 | | 2.05 | 1.63 | 1.55 |
| | 2.00 | | 1.78 | 1.64 | 1.30 |
| | 3.00 | | 1.27 | 1.47 | 1.11 |
| | 4.00 | | 0.98 | 1.39 | 0.98 |
| | 5.00 | | 1.01 | 1.21 | 0.89 |
| | 6.00 | | 0.90 | 1.06 | 0.84 |
| | 6.25 | | 1.17 | | 0.88 |
| | 6.50 | | 1.88 | | 1.06 |
| | 7.00 | | 2.12 | | 1.20 |
| | 7.50 | | 2.24 | | 1.15 |
| | 8.00 | 1.32 | 2.01 | 0.97 | 1.03 |
| | 9.00 | | 1.52 | | 0.90 |
| | 10.0 | 1.32 | 1.24 | 0.85 | 0.84 |
| | 11.0 | | 1.11 | | 0.74 |
| | 12.0 | 1.18 | 0.96 | 0.79 | 0.70 |

TABLE 21
Mean Single-Dose Pharmacokinetic Results

| | Controlled | | Immediate | |
|---|---|---|---|---|
| | Release 20 mg | | Release 10 mg | |
| | oxymor- | 6-OH- | | 6-OH-oxy- |
| | phone | oxymorphone | oxymorphone | morphone |
| AUC₍ₒ₋ₜ₎ | 14.74 | 11.54 | 7.10 | 5.66 |
| AUC_{(o-inf)} | 15.33 | 16.40 | 7.73 | 8.45 |
| Cₘₐₓ(ng/ml) | 1.12 | 0.68 | 1.98 | 1.40 |
| Tₘₐₓ(hr) | 5.00 | 2.00 | 0.50 | 0.50 |
| T½(hr) | 9.25 | 26.09 | 10.29 | 29.48 |

Parent 6-OH oxymorphone AUC₍₀₋ₜ₎ values were lower than the parent compound after administration of either dosage form, but the AUC_{(o-inf)} values are slightly higher due to the longer half-life for the metabolite. This relationship was similar for both the immediate-release (IR) and controlled release (CR) dosage forms. As represented by the average plasma concentration graph, the CR dosage form has a significantly longer time to peak oxymorphone concentration and a lower peak oxymorphone concentration. The 6-OH oxymorphone peak occurred sooner than the parent peak following the CR dosage form, and simultaneously with the parent peak following the IR dosage form.

It is important to note that while the present invention is described and exemplified using 20 mg tablets, the invention may also be used with other strengths of tablets. In each strength, it is important to note how a 20 mg tablet of the same composition (except for the change in strength) would act. The blood plasma levels and pain intensity information are provided for 20 mg tablets, however the present invention is also intended to encompass 5 to 80 mg controlled release tablets. For this reason, the blood plasma level of oxymorphone or 6-hydroxyoxymorphone in nanograms per milliliter of blood, per mg oxymorphone (ng/mg·ml) administered is measured. Thus at 0.02 ng/mg·ml, a 5 mg tablet should produce a minimum blood plasma concentration of 0.1 ng/ml. A stronger tablet will produce a higher blood plasma concentration of active molecule, generally proportionally. Upon administration of a higher dose tablet, for example 80 mg, the blood plasma level of oxymorphone and 6-OH oxymorphone may more than quadruple compared to a 20 mg dose, although conventional treatment of low bioavailability substances would lead away from this conclusion. If this is the case, it may be because the body can only process a limited amount oxymorphone at one time. Once the bolus is processed, the blood level of oxymorphone returns to a proportional level.

It is the knowledge that controlled release oxymorphone tablets are possible to produce and effective to use, which is most important, made possible with the high bioavailability of oxymorphone in a controlled release tablet. This also holds true for continuous periodic administration of controlled release formulations. The intent of a controlled release opioid formulation is the long-term management of pain. Therefore, the performance of a composition when administered periodically (one to three times per day) over several days is important. In such a regime, the patient reaches a “steady state” where continued administration will produce the same results, when measured by duration of pain relief and blood plasma levels of pharmaceutical. Such a test is referred to as a “steady state” test and may require periodic administration over an extended time period ranging from several days to a week or more. Of course, since a patient reaches steady state in such a test, continuing the test for a longer time period should not affect the results. Further, when testing blood plasma levels in such a test, if the time period for testing exceeds the interval between doses, it is important the regimen be stopped after the test is begun so that observations of change in blood level and pain relief may be made without a further dose affecting these parameters.

Study 5—Controlled Release 40 mg vs Immediate Release 4.times.10 mg under Fed and Fasting Conditions

The objectives of this study were to assess the relative bioavailability of oxymorphone from oxymorphone controlled release (40 mg) compared to oxymorphone immediate release (4.times.10 mg) under both fasted and fed conditions, and to determine the effect of food on the bioavailability of oxymorphone from the controlled release formulation, oxymorphone CR, and from the immediate release formulation, oxymorphone IR.

This study had a single-center, open-label, analytically blinded, randomized, four-way crossover design. Subjects randomized to Treatment 5A and Treatment 5C, as described below, were in a fasted state following a 10-hour overnight fast. Subjects randomized to Treatment 5B and Treatment 5D, as described below, were in the fed state, having had a high fat meal, completed ten minutes prior to dosing. There was a 14-day washout interval between the four dose administrations. The subjects were confined to the clinic during each study period. Subject assigned to receive Treatment 5A and Treatment 5B were discharged from the clinic on Day 3 following the 48-hour procedures, and subjects assigned to receive Treatment 5C and Treatment 5D were discharged from the clinic on Day 2 following the 36-hour procedures. On Day 1 of each study period the subjects received one of four treatments:

Treatments 5A and 5B: Oxymorphone controlled release 40 mg tablets from Table 2. Subjects randomized to Treatment 5A received a single oral dose of one 40 mg oxymorphone controlled release tablet taken with 240 ml of water after a 10-hour fasting period. Subjects randomized to Treatment 5B received a single oral dose of one 40 mg oxymorphone controlled release tablet taken with 240 ml of water 10 minutes after a standardized high fat meal.

Treatments 5C and 5D: Immediate release tablet (IR)4.times.10 mg Oxymorphone. Subjects randomized to Treatment 5C received a single oral dose of 4.times.10 mg oxymorphone IR tablet taken with 240 ml of water after a 10-hour fasting period. Subjects randomized to Treatment 5D received a single oral dose of 4.times.10 mg oxymorphone IR tablet taken with 240 ml of water 10 minutes after a standardized high-fat meal.

A total of 28 male subjects were enrolled in the study, and 25 subjects completed the study. A total of 28 subjects received at least one treatment. Only subjects who completed all 4 treatments were included in the summary statistics and statistical analysis.

Blood samples (7 ml) were collected during each study period at the 0 hour (predose), and at 0.25, 0.5, 0.75, 1.0, 1.5, 2, 3, 4, 5, 6, 8, 10, 12, 24, 36, 48, 60, and 72 hours post-dose (19 samples) for subjects randomized to all Treatments.

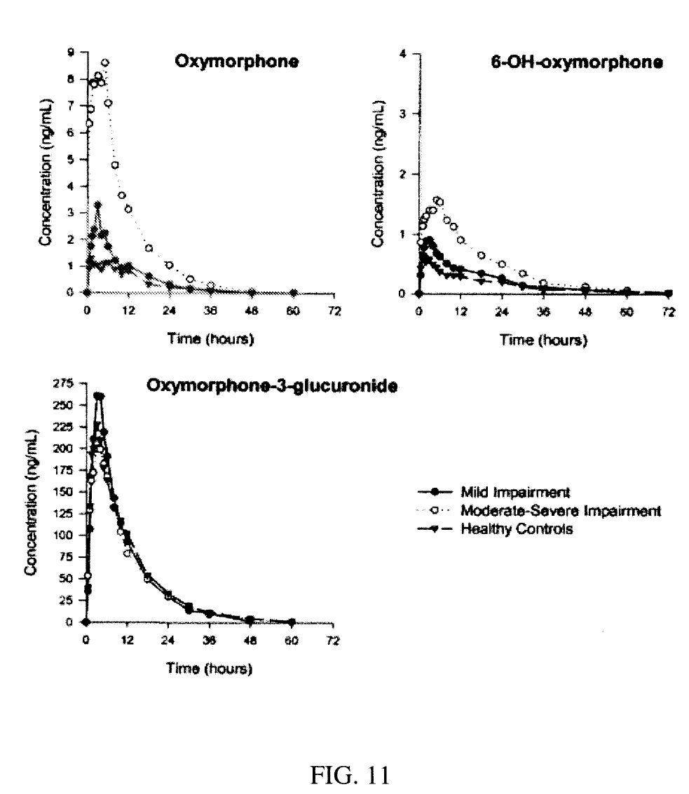

The mean oxymorphone plasma concentration versus time curves for Treatments 5A, 5B, 5C, and 5D are presented in FIG. 11. The data is contained in Table 22. The arithmetic means of the plasma oxymorphone pharmacokinetic parameters and the statistics for all Treatments are summarized in Table 23.

TABLE 22
Mean Plasma Concentration vs. Time (ng/ml)

| | Treatment | Treatment | Treatment | Treatment |
|---|---|---|---|---|
| Time (hr) | 5A | 5B | 5C | 5D |
| 0 | 0.00 | 0.00 | 0.00 | 0.00 |
| 0.25 | 0.47 | 0.22 | 3.34 | 1.79 |
| 0.50 | 1.68 | 0.97 | 7.28 | 6.59 |
| 0.75 | 1.92 | 1.90 | 6.60 | 9.49 |
| 1 | 2.09 | 2.61 | 6.03 | 9.91 |
| 1.5 | 2.18 | 3.48 | 4.67 | 8.76 |
| 2 | 2.18 | 3.65 | 3.68 | 7.29 |
| 3 | 2.00 | 2.86 | 2.34 | 4.93 |
| 4 | 1.78 | 2.45 | 1.65 | 3.11 |
| 5 | 1.86 | 2.37 | 1.48 | 2.19 |
| 6 | 1.67 | 2.02 | 1.28 | 1.71 |
| 8 | 1.25 | 1.46 | 0.92 | 1.28 |
| 10 | 1.11 | 1.17 | 0.78 | 1.09 |
| 12 | 1.34 | 1.21 | 1.04 | 1.24 |
| 24 | 0.55 | 0.47 | 0.40 | 0.44 |
| 36 | 0.21 | 0.20 | 0.16 | 0.18 |
| 48 | 0.06 | 0.05 | 0.04 | 0.05 |
| 60 | 0.03 | 0.01 | 0.01 | 0.01 |
| 72 | 0.00 | 0.00 | 0.00 | 0.00 |

TABLE 23
Pharmacokinetic Parameters of Plasma Oxymorphone for Study 5

| | Treatment | | Treatment | | Treatment | | Treatment | |
|---|---|---|---|---|---|---|---|---|
| | 5A | | 5B | | 5C | | 5D | |
| | Mean | SD | Mean | SD | Mean | SD | Mean | SD |
| Cₘₐₓ | 2.79 | 0.84 | 4.25 | 1.21 | 9.07 | 4.09 | 12.09 | 5.42 |
| Tₘₐₓ | 2.26 | 2.52 | 1.96 | 1.06 | 0.69 | 0.43 | 1.19 | 0.62 |
| AUC₍ₒ₋ₜ₎ | 35.70 | 10.58 | 38.20 | 11.04 | 36.00 | 12.52 | 51.35 | 20.20 |
| AUC_{(o-inf)} | 40.62 | 11.38 | 41.17 | 10.46 | 39.04 | 12.44 | 54.10 | 20.26 |
| T_{1/2el} | 12.17 | 7.57 | 10.46 | 5.45 | 11.65 | 6.18 | 9.58 | 3.63 |

The relative bioavailability calculations are summarized in Tables 24 and 25.

TABLE 24
Relative Bioavailability Determination Based on AUC_{(o-inf)}

| Fᵣₑₗ (5D vs. 5C) | Fᵣₑₗ (5B vs. 5A) |
|---|---|
| 1.3775 | 1.0220 |

TABLE 25
Relative bioavailability Determination Based on AUC₍ₒ₋₂₄₎

| Fᵣₑₗ (5D vs. 5C) | Fᵣₑₗ (5B vs. 5A) |
|---|---|
| 1.4681 | 1.0989 |

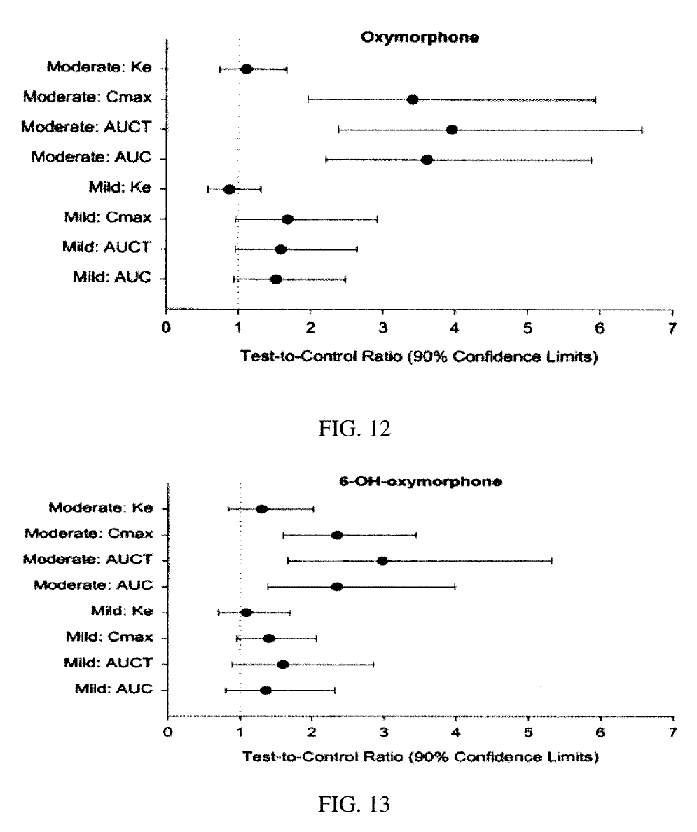

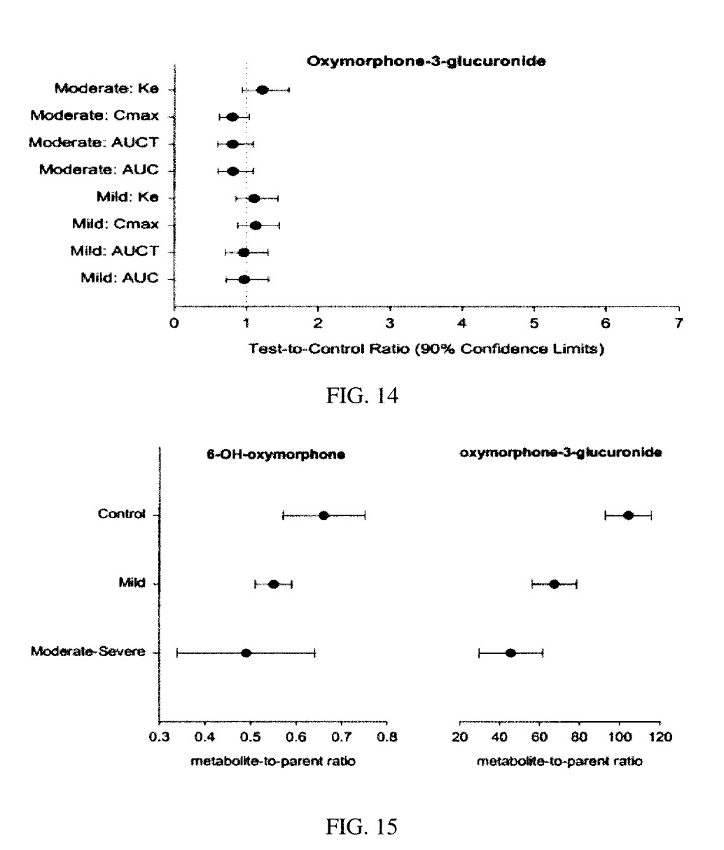

The mean 6-OH oxymorphone plasma concentration versus time curves for Treatments 5A, 5B, 5C, and 5D are presented in FIG. 12. The data is contained in Table 26.

TABLE 26
Mean Plasma Concentration vs. Time (ng/ml)
6-Hydroxyoxmorphone

| | Treatment | Treatment | Treatment | Treatment |
|---|---|---|---|---|
| Time (hr) | 5A | 5B | 5C | 5D |
| 0 | 0.00 | 0.00 | 0.00 | 0.00 |
| 0.25 | 0.27 | 0.05 | 2.36 | 0.50 |
| 0.05 | 1.32 | 0.31 | 5.35 | 1.98 |
| 0.75 | 1.37 | 0.59 | 4.53 | 2.97 |
| 1 | 1.44 | 0.82 | 3.81 | 2.87 |
| 1.5 | 1.46 | 1.09 | 2.93 | 2.58 |
| 2 | 1.46 | 1.28 | 2.37 | 2.29 |
| 3 | 1.39 | 1.14 | 1.69 | 1.72 |
| 4 | 1.25 | 1.14 | 1.33 | 1.26 |
| 5 | 1.02 | 1.00 | 1.14 | 1.01 |
| 6 | 0.93 | 0.86 | 0.94 | 0.86 |
| 8 | 0.69 | 0.72 | 0.73 | 0.77 |
| 10 | 0.68 | 0.67 | 0.66 | 0.75 |
| 12 | 0.74 | 0.66 | 0.70 | 0.77 |
| 24 | 0.55 | 0.52 | 0.54 | 0.61 |
| 36 | 0.23 | 0.30 | 0.28 | 0.27 |
| 48 | 0.18 | 0.20 | 0.20 | 0.19 |
| 60 | 0.09 | 0.10 | 0.09 | 0.09 |
| 72 | 0.06 | 0.06 | 0.04 | 0.05 |

TABLE 27
Pharmacokinetic Parameters of Plasma
6-Hydroxyoxymorphone for Study 5

| | Treatment | | Treatment | | Treatment | | Treatment | |
|---|---|---|---|---|---|---|---|---|
| | 5A | | 5B | | 5C | | 5D | |
| | Mean | SD | Mean | SD | Mean | SD | Mean | SD |
| Cₘₐₓ | 1.88 | 0.69 | 1.59 | 0.63 | 6.41 | 3.61 | 3.79 | 1.49 |
| Tₘₐₓ | 1.48 | 1.18 | 2.73 | 1.27 | 0.73 | 1.47 | 1.18 | 0.74 |
| AUC₍ₒ₋ₜ₎ | 28.22 | 10.81 | 26.95 | 11.39 | 33.75 | 10.29 | 32.63 | 13.32 |
| AUC_{(o-inf)} | 33.15 | 11.25 | 32.98 | 10.68 | 37.63 | 17.01 | 36.54 | 13.79 |
| T_{1/2el} | 17.08 | 7.45 | 21.92 | 8.41 | 16.01 | 6.68 | 16.21 | 7.42 |

### Example 5

Introduction

Oxymorphone HCl is highly metabolized principally in the liver and undergoes conjugation with glucuronic acid to form both active and inactive products. Cone et al. reported on the urinary metabolites of oxymorphone following administration of a 10 mg oral dose in 10 healthy subjects. The concentrations of oxymorphone and 6-OH-oxymorphone were measured before and after hydrolysis of the urine. On average, 49% of the administered dose was recovered in the urine over a 120-hour collection interval. The majority of the recovery (˜42% of the dose, 82% of the amount recovered) occurred in the first 24 hours. Very little unchanged oxymorphone was recovered in the urine (1.9%, range 0.3% to 0.5%). Conjugated oxymorphone accounted for an average of 44.1% (range 27.2% to 63.1%) of the administered dose. Urinary recovery of the 6-OH metabolite accounted for approximately 3% of the dose (˜0.3% free and 2.6% conjugated). The identity, with respect to type (e.g., glucuronic acid) or position (e.g., 3- to 14-), of the oxymorphone conjugates was not identified. None of the animal species (rat, dog, guinea pig, or rabbit) had a urinary metabolite profile that was similar to man.

Study Objectives

The objective of this study is to determine the pharmacokinetics and metabolism of EN3202 (oxymorphone hydrochloride extended-release) tablets in patients with hepatic impairment under fasting conditions.

Methods

Clinical Study Design and Conduct

This study employed a single-dose, parallel group design in 12 subjects with chronic hepatic cirrhosis (6 in Child-Pugh Class A and 6 in Child-Pugh Class B or worse) and 12 healthy controls matched for age, weight, and gender. Each subject received a single 20 mg dose of ER oxymorphone. The oxymorphone ER tablets administered were according to the now-available commercial formula of Opana® 20 mg strength, which also contains the inactive ingredients hypromellose, iron oxide black, methylparaben, propylene glycol, silicified microcrystalline cellulose, sodium stearyl fumarate, TIMERx®-N, titanium dioxide and triacetin, FD&C blue No. 1, FD&C yellow No. 6, and FD&C yellow No. 10. Naltrexone 50 mg was administered on the evening prior to administration of the ER oxymorphone dose. Plasma and urine samples were collected at specified intervals for six days after the dose to determine oxymorphone and metabolite concentrations. Study participants were housed in the clinical research facility throughout the treatment period, beginning on the evening prior to administration of the test medication and extending until collection of the 120-hour blood sample and urine samples. While in the clinic, the subjects were to refrain from strenuous physical activity.

Overall Study Design

The study procedures are outlined in the following table (Table 28).

TABLE 28
Schedule of Study Evaluations

| | Phase | | | | | | | |
|---|---|---|---|---|---|---|---|---|
| | Screening | | | | | | | |
| | VISIT | | | | | | | |
| | NUMBER | | | Treatment | | | | |
| | 1 | 2 | | Period | | | | |
| | DAY | | | | | | | |
| | −14 | −1 | 1 | 2 | 3 | 4 | 5 | 6 |
| Medical/Medication History | X | | | | | | | |
| Informed Consent | X | | | | | | | |
| Assessment of Eligibility | X | X | | | | | | |
| Physical Examination | X | | | | | | | X |
| 12-Lead Electrocardiogram | X | | | | | | | |
| Clinical Laboratory Tests | X | | | | | | | X |
| Vital Signs | X | X | X | X | X | X | X | X |
| Body Weight | X | X | | | | | | |
| Urine Drug Screen | X | X | | | | | | |
| Naltrexone Dose | | X | | | | | | |
| ER oxymorphone Dose | | | X | | | | | |
| Plasma Samples | | | (a) | (a) | (a) | (a) | (a) | (a) |
| Urine Collection | | | (b) | (b) | (b) | (b) | (b) | (b) |
| Assessment of Adverse | | | | | | | | |
| Events | | | X | X | X | X | X | X |

(a) Plasma sample times: 0 (pre-dose), 0.5, 1.0, 1.5, 2.0, 3.0, 4.0, 5.0, 6.0, 8.0, 10.0, 12.0, 18.0, 24, 30, 36, 48, 60, 72, 84, 96, 108, and 120 hours after dose administration.
(b) Urine collection intervals: 0 (pre-dose), 0-12, 12-24, 24-48, 48-72, 72-96, and 96-120 hours after administration of the test medication.

A series of screening evaluations were performed in order to determine whether prospective study participants met the selection criteria for the trial. Screening evaluations were performed within a 14-day period, prior to receiving the study medication. Screening evaluations consisted of a medical history, a review of systems, medication history, physical examination, vital signs, weight, 12-lead electrocardiogram (ECG), laboratory evaluations (standard hematology and serum chemistry panels, and urinalysis), and urine drug screen. Additional vital signs determination, including pulse, blood pressure, and respiratory rate were obtained at 24, 48, 72, 96, and 120 hours after administration of the test medication. Blood pressure and pulse were obtained with the subject in the sitting position after sitting for 5 minutes. Additional vital signs obtained during the course of the study, when clinically indicated, were to be supplied to the sponsor. Laboratory evaluations, physical examination and vitals signs measurements were repeated at the conclusion of the treatment period. Screening tests for hepatitis and HIV infection were obtained at the Screening visit only.

Any significant abnormalities were to be fully investigated. Whenever possible, the etiology of the abnormal findings were documented on the case report form (CRF). Laboratory results with significantly abnormal values were to be repeated for verification. Any significant laboratory abnormalities that were either serious or unexpected were to be promptly reported to the study monitor. Any additional relevant laboratory results obtained by the investigator during the course of the study were to be supplied to the sponsor.

During the study period, study participants reported to the clinic on the evening prior to dose administration and remained in the clinic until released by the investigator following the final blood draw on Study Day 6 and the study evaluations to be conducted at the end of study. Final study evaluations included physical exam, laboratory evaluations, vital signs, and assessment of adverse experiences.

At the screening visit, subjects were informed not to take any medications (Rx or OTC) until the study began. Hepatically impaired subjects could take approved prescribed concomitant medications. The investigator informed each prospective subject of the nature of the study, explained the potential risks, and obtained written informed consent from the subject prior to performing any procedures involving more than minimal risk and prior to the administration of study medication. Participants had to meet all of the study entrance criteria to continue in the study.

Hepatically impaired subjects of either sex were enrolled in the two Child-Pugh classes, six in Class A and six in Class B or worse, as they became available. The 12 healthy volunteers were matched to hepatically impaired subjects by age, weight, and gender.

This was a single-dose trial. As a result, any subject who received the dose of test medication should have remained in the trial and completed all required tests and evaluations. Reasons for premature discontinuation are discussed below.
The subject withdrew their consent for further participation;
The investigator determined that continued participation in the trial placed the subject at unacceptable risk; and
The investigator determined that the subject required medical treatment that could not be administered at the study facility.

Subjects who withdrew from the study prior to completion of the study evaluations and blood samples scheduled for 72 hours following dose administration could be replaced. Up to 28 participants could be enrolled to allow for completion of 24 subjects. Plasma samples were not to be analyzed for subjects who discontinued from the trial prior to collection of the 72-hour blood sample. If a subject withdrew from the study, the investigator was to contact the monitor to discuss the necessity of replacement; the decision was to be made prior to analysis of the plasma samples. The replacement subject was to match the population of the subject who was withdrawn (i.e., healthy control or hepatically impaired).

The date the subject was withdrawn from the study and the reason for discontinuation was to be recorded on the CRF. When a subject was withdrawn from the study (regardless of the reason), all evaluations required at the final study visit were to be performed.

Selection of the Study Population

Participants were selected from hepatically impaired and healthy adult volunteers in the general geographic area of the clinical research facility.

Twelve healthy volunteers had to meet all of the following criteria for inclusion into the study:
Healthy males or nonpregnant females aged 18 to 70 years of age. Female patients of childbearing potential must have had a negative serum β-hCG level consistent with nongravid state at the screening visit and agreed to use an appropriate method of contraception;
Had body weights not less than 110 lb and were within 30% of the Metropolitan Life Insurance Company's standards dated 1983 (see Appendices B and C of the protocol);
Able to communicate effectively with the study personnel;
Able to match with a hepatically-impaired volunteer for gender and as closely as possible with regard to body weight (±15%) and age (±5 years);
No significant disease or abnormal laboratory values as determined by medical history, physical examination, or laboratory evaluations, conducted at the Screening visit or on admission to the clinic;
Had a normal 12-lead ECG, without any clinically significant abnormalities of rate, rhythm, or conduction;
Had not consumed alcoholic beverages within 72 hours prior to administration of the first dose of study medication; and
Was adequately informed of the nature and risks of the study and gave written informed consent prior to receiving study medication.

Twelve male or female subjects with chronic hepatic cirrhosis had to meet the following criteria for inclusion into the study:
Male or nonpregnant female 18 to 70 years of age. Female patients of childbearing potential must have had a negative serum β-hCG level consistent with nongravid state at the screening visit and agreed to use an appropriate method of contraception;
Had a diagnosis of chronic (for more than six months), stable (no acute episodes of illness within the previous two months due to deterioration of hepatic function) hepatic insufficiency with features of cirrhosis due to any etiology;
Had a total score on the Child-Pugh scale ranging from 5 to 14 at screening. A minimum of six subjects must have had Grade B or worse hepatic impairment (total score of 7 to 14);
Had body weights not less than 110 lb and within 30% of the Metropolitan Life Insurance Company's standards dated 1983 (see Appendices B and C of the protocol);
Were able to communicate effectively with study personnel;
Had a prothrombin time<10 seconds over control;
Had a hemoglobin concentration≧10 g/dL;
Had a platelet count>50,000/μ;
Had a normal 12-lead electrocardiogram, without any clinically significant abnormalities of rate, rhythm, or conduction;
Had not consumed alcoholic beverages within 72 hours prior to administration of the first dose of study medication; and
Were adequately informed of the nature and risks of the study and gave written informed consent prior to receiving study medication.

Healthy participants to whom any of the following applied were to be excluded from the study:
Known hypersensitivity or allergy to oxymorphone or naltrexone;
Any disease or condition (medical or surgical) which might compromise the hematologic, cardiovascular, pulmonary, renal, gastrointestinal, hepatic, or central nervous system, or other conditions that may interfere with the absorption, distribution, metabolism, or excretion of study drug, or would place the subject at increased risk;
The presence of abnormal laboratory values which were considered clinically significant. In addition, no subject with tests of liver function (SGOT, SGPT) above 1.25 times the upper limit of normal, total bilirubin above the upper limit of normal, serum creatinine above the upper limit of normal, or hematologic function (hemoglobin, hematocrit, white blood cells, or platelets) below the lower limit of normal were to be admitted to the study;
Positive screen for Hepatitis B consisting of HBsAg (Hepatitis B Surface Antigen) or HIV;
Received any investigational drug within a period of 30 days prior to enrollment in the study or any prescription drug therapy within 2 weeks of initiation of the study. This exclusion was extended to 4 weeks for any drugs known to affect hepatic drug metabolism. No non-prescription (OTC) drugs could be taken within 24 hours of admission into the study;
A positive urine drug screen including alcohol, cocaine, THC, barbiturates, amphetamines, benzodiazepines, and opiates;
Any history of alcohol abuse, illicit drug use, significant mental illness, physical dependence to any opioid, or any history of drug abuse or addiction;
A history of difficulty with donating blood; or
Received the study medication previously.

Hepatically impaired participants to whom any of the following applied were to be excluded from the study:
Known hypersensitivity or allergy to oxymorphone or naltrexone;
Had any disease or condition (medical or surgical) that might compromise the hematologic, cardiovascular, pulmonary, renal, gastrointestinal, or central nervous system, other than those relating to liver disease;
The presence of significant abnormalities on pre-study screening clinical examination or laboratory measurements, other than those related to liver disease, carried out about 2 weeks prior to commencement of the study;
Was receiving oral contraceptive and/or hormone-replacement therapy;
Had a positive screen for either Hepatitis B (Hepatitis B Surface Antigen) or HIV;
Received an investigational drug within a period of 30 days prior to enrollment in the study, or used an unacceptable concomitant medication;
Had a positive urine drug screen including ethanol, cocaine, THC, barbiturates, amphetamines, benzodiazepines, and opiates;
Any history of alcohol abuse illicit drug use, had significant mental illness, physical dependence to any opioid, or any history of drug abuse or addiction;
Had a history of difficulty donating blood; or
Had received the study medication previously.

Hepatically impaired subjects of either sex were enrolled in the two Child-Pugh classes, six in Class A and six in Class B or worse, as they became available. The 12 healthy volunteers were matched to hepatically impaired subjects by age, weight, and gender.

Study Medications

All study medications were supplied by the sponsor. Medications utilized in this trial were:
EN3202 (oxymorphone hydrochloride extended-release) tablets 20 mg;
ReVia® (naltrexone hydrochloride) tablets 50 mg.
   In order to protect the subjects from potential opioid related adverse events, the opioid antagonist ReVia® (naltrexone hydrochloride) was administered at a dosage of 50 mg once, on the evening prior to administration of the first oxymorphone dose (Day-1). A single 20 mg dose EN3202 was administered following an overnight fast (from approximately 22:00 on the evening prior to dose administration) at approximately 08:00, and participants were not allowed to eat until at least four hours after the dose.

All doses were administered with 240 mL of ambient water. Participants were instructed to drink all of the water.

The study was neither blinded nor randomized.

Safety Assessments

Although a standard 12-lead ECG was only scheduled at Screening, additional ECGs were to be obtained if clinically indicated. A follow-up ECG was to be obtained if any significant adverse events were detected after dose administration that would warrant an ECG. Any additional relevant data obtained by the investigator during the course of the study was to be supplied to the sponsor.

Routine vital signs, including pulse, respirations, blood pressure and temperature were obtained in conjunction with the physical examination and just prior to administration of the test medication. Additional vital signs determination, including pulse, blood pressure, and respiratory rate were obtained at 24, 48, 72, 96, and 120 hours after administration of the test medication.

Blood pressure and pulse were obtained with the subject in the sitting position after sitting for 5 minutes. Additional vital signs obtained during the course of the study, when clinically indicated, were to be supplied to the sponsor. A battery of clinical laboratory tests (including hematology, serum chemistry, and urinalysis) was obtained at screening and on the last day of the study (Day 6). A physical examination was performed at screening and on the last day of the study (Day 6).

Each subject was carefully monitored for the development of any adverse experiences. This information was obtained in the form of non-leading questions (e.g., “How are you feeling?”) and from signs and symptoms detected during each examination, observations of the study personnel or spontaneous reports from the subjects.

Analytical Methods

A total of five (5) validated LC/MS/MS methods were utilized to measure the concentrations of oxymorphone, 6-OH-oxymorphone, and oxymorphone-3-glucuronide in plasma and urine samples. The methods include: oxymorphone and 6-OH-oxymorphone in plasma (M001005.01); oxymorphone-3-glucuronide in plasma (M001107.00); oxymorphone-3-glucuronide in urine (M001106.00); oxymorphone and 6-OH-oxymorphone in urine (M001007.00); and “Total” oxymorphone and 6-OH-oxymorphone in urine (M001001.00). In all methods, the internal standards are d3-oxymorphone, d3-6-OH-oxymorphone, and d3-oxymorphone-3-glucuronide for oxymorphone, 6-OH-oxymorphone, and oxymorphone-3-glucuronide, respectively. The methods for simultaneous determination of oxymorphone and 6-OH-oxymorphone utilize liquid-liquid extraction of plasma or urine; the method for oxymorphone-3-glucuronide utilizes solid phase extraction. The method for “Total” oxymorphone and 6-OH-oxymorphone includes incubation of the urine sample with β-glucuronidase for two hours at 50° C. prior to addition of the internal standard and extraction. Validation results are summarized in the following tables:

TABLE 29
Summary of Method Validation Results for Plasma Analytes

| Parameter | OXM | 6-OH-OXM | OXM-3-G |
|---|---|---|---|
| Standard Concentrations (ng/mL) | 0.1, 0.2, 0.5, 1, 5, 10, | 0.1, 0.2, 0.5, 1, 5, 10, | 5, 12.5, 25, 50, 125, |
| | 18, 20 | 18, 20 | 200, 250 |
| QC Concentrations (ng/mL) | 0.3, 6, 14 | 0.3, 6, 14 | 15, 75, 180 |
| Linearity (mean r) | 0.9994 | 0.9987 | 0.9982 |
| Linear Range (ng/mL) | 0.1-20 | 0.1-20 | 5-250 |
| LOQ (ng/mL) | 0.1 | 0.1 | 5.0 |
| Intra-day Precision (% CV)* | 1.43-3.93 | 2.12-7.87 | 1-39-6.79 |
| Intra-day Accuracy (% Actual)* | 94.33-96.56 | 98.17-102.56 | 101.00-105.31 |
| Inter-day Precision (% CV)* | 2.86-7.77 | 3.83-7.74 | 3.85-5.53 |
| Inter-day Accuracy (% Actual)* | 97.31-99.36 | 100.36-101.70 | 98.99-102.36 |
| Recovery (%) | 53.98 | 22.96 | 79.30 |

*precision and accuracy results based on QC samples
OXM = oxymorphone
6-OH-OXM = 6-OH-oxymorphone
OXM-3-G = oxymorphone-3-glucuronide

TABLE 30
Summary of Method Validation Results for Urine Analytes

| Parameter | OXM | 6-OH-OXM | OXM-3-G |
|---|---|---|---|
| Standard Concentrations (ng/mL) | 1, 2.5, 10, 25, 50, | 1, 2.5, 10, 25, 50, | 10, 25, 100, 250, 500, |
| | 100, 150, 200 | 100, 150, 200 | 1000, 1500, 2000 |
| QC Concentrations (ng/mL) | 1.5, 60, 140 | 1.5, 60, 140 | 30, 600, 1400 |
| Linearity (mean r) | 0.9994 | 0.9988 | 0.9990 |
| Linear Range (ng/mL) | 1-200 | 1-200 | 10-2000 |
| LOQ (ng/mL) | 1.0 | 1.0 | 10.0 |
| Intra-day Precision (% CV)* | 2.00-7.26 | 3.04-4.73 | 4.63-6.02 |
| Intra-day Accuracy (% Actual)* | 101.89-103.49 | 99.56-103.43 | 99.41-103.59 |
| Inter-day Precision (% CV)* | 3.18-7.04 | 3.73-7.86 | 4.55-6.17 |
| Inter-day Accuracy (% Actual)* | 98.92-102.33 | 98.70-100.65 | 97.50-99.26 |
| Recovery (%) | 62.92 | 67.19 | 78.51 |

*precision and accuracy results based on QC samples
OXM = oxymorphone
6-OH-OXM = 6-OH-oxymorphone
OXM-3-G = oxymorphone-3-glucuronide

TABLE 31
Summary of Method Validation Results for “Total” Oxymorphone
and 6-OH-oxymorphone in Urine

| Parameter | OXM | 6-OH-OXM |
|---|---|---|
| Standard Cons. (ng/mL) | 2, 5, 20, 50, 100, | 2, 5, 20, 50, 100, |
| | 200, 300, 400 | 200, 300, 400 |
| QC Conc. (ng/mL) | 6, 140, 280 | 6, 140, 280 |
| Linearity (mean r) | 0.9983 | 0.9987 |
| Linear Range (ng/mL) | 2-400 | 2-400 |
| LOQ (ng/mL) | 2.0 | 2.0 |
| Intra-day Precision (% CV)* | 2.81-5.58 | 2.05-4.91 |
| Intra-day Accuracy (% Actual)* | 100.23-102.01 | 101.66-106.02 |
| Inter-day Precision (% CV)* | 3.28-6.49 | 5.38-7.98 |
| Inter-day Accuracy (% Actual)* | 96.92-101.76 | 99.99-103.75 |
| Recovery | 85.76 | 31.14 |

*precision and accuracy results based on QC samples
OXM = oxymorphone
6-OH-OXM = 6-OH-oxymorphone
OXM-3-G = oxymorphone-3-glucuronide

Pharmacokinetic and Statistical Methods

This was a parallel group trial designed to enroll 12 subjects with chronic hepatic impairment due to cirrhosis and 12 healthy control subjects matched for gender and age.

Samples of venous blood were obtained in a 7 mL EDTA tube just prior to dose administration (time 0), and at 0.5, 1.0, 1.5, 2.0, 3.0, 4.0, 5.0, 6.0, 8.0, 10.0, 12.0, 18.0, 24.0, 30.0, 36.0, 48.0, 60.0, 72.0, 84.0, 96.0, 108.0, and 120 hours after dose administration.

Urine samples were obtained for 120 hours after dose administration. Subjects were to be instructed to void just prior to administration of the test medication (time 0) and all urine was collected during the intervals of 0-12, 12-24, 24-48, 48-72, 72-96, and 96-120 hours after administration of the test medication.

Calculation of Pharmacokinetic Variables

Pharmacokinetic parameters were calculated using model-independent methods. The definitions and methods of calculation are summarized in the following table:

TABLE 32
Defination of Pharmacokinetic Variables

| Variable | Defination |
|---|---|
| Cₘₐₓ | Maximum plasma concentration; the highest concentration |
| | observed during a dosage interval. |
| Tₘₐₓ | The time that Cₘₐₓ was observed. |
| Cₜ | The last measured plasma concentration; the last |
| | concentration above the lower limit of quantitation) |
| | following a dose. |
| λ_{z}(Ke) | The terminal elimination rate constant; calculated using |
| | linear regression on the terminal portion of the |
| | Ln-concentration versus time curve. |
| T½ | Terminal elimination half-life; calculated as 0.693/λ_{z}. |
| AUCT | Area under the concentration versus time curve from time 0 |
| | to the last measured concentration (Cₜ); calculated using |
| | the trapezoidal rule. |
| AUC | Area under the concentration versus time curve from time 0 to |
| | infinity; calculated as AUCT + Cₜ/λ_{z}. |
| CL | Total systemic clearence (following i.v. administration); |
| | calulated as Dose (i.v.)/AUC. |
| CL/Fₒ | Oral clearence; calculated as Dose (p.o)/AUC. |

Statistical Methods

All pharmacokinetic results are summarized using appropriate descriptive statistics. Following log-transformation (natural log), AUC, AUCT, Cₘₐₓ and λ_{z} results were be compared between treatment groups using a 90% confidence interval approach. One-way ANOVA was conducted using the model PKVAR=group, where group was mild hepatic impairment, moderate-severe hepatic impairment, or healthy control. Point estimates of the impaired versus control group differences and the corresponding 90% confidence limits were constructed. The relationship between measures of hepatic disease (e.g., serum albumin, bilirubin, prothrombin time) and oxymorphone oral clearance was explored.

The frequency of adverse experiences (AEs) were tabulated by MedDRA term and body system. The incidence of AEs is compared across treatment groups using an appropriate non-parametric statistic. The maximum intensity and frequency of AEs are summarized by treatment group. A new-onset AE is defined as an AE that was not present prior to treatment with study medication but appeared following treatment, or was present at treatment initiation but worsened during treatment. An AE that was present at treatment initiation but resolved and then reappeared while the patient was on treatment is a new-onset AE (regardless of the intensity of the AE when the treatment was initiated).

All vital sign measurements are summarized by mean values and changes from baseline. Changes from baseline were be analyzed across treatment groups using an appropriate parametric statistic.

Results

Disposition of Subjects

Twenty-four (24) participants, 12 healthy, and 12 hepatically impaired were enrolled and received treatment; all 24 completed the trial. The study population consisted of 16 men and 8 women ranging from 44 to 73 years of age. The hepatically impaired group included 6 subjects in Child-Pugh Class A, 5 subjects in Child-Pugh Class B, and 1 subject in Child-Pugh Class C. Within this report, subjects with hepatic impairment are classified as having “mild” impairment (Child-Pugh Class A) or “moderate-severe” impairment (Child-Pugh Class B and C). All three treatment groups were well matched for age, gender, height, and body weight (Table 33).

TABLE 33
Summary of Demographic Characteristics

| | | Moderate-Serve | |
|---|---|---|---|
| | Mild Impairment | Impairment | Healthy Controls |
| Number | 6 | 6 | 12 |
| Males | 4 | 4 | 8 |
| Females | 2 | 2 | 4 |
| Age (years) | 55.3 (3.96) | 52.5 (4.51) | 54.4 (2.53) |
| Height (in) | 66.3 (1.74) | 65.8 (1.35) | 65.7 (1.05) |
| Weight (Ibs) | 175.5 (13.7) | 177.2 (14.4) | 168.1 (7.45) |

Mean (SE)

Protocol Deviations

There were no deviations in the collection times for pharmacokinetic samples. Two (2) subjects with hepatic impairment and one (1) healthy control (Numbers 001 age 73, 010 age 71, and 013 age 17) received waivers of the age requirement. Three subjects had clinically insignificant deviations from the laboratory reference range or protocol criteria at screening and were admitted. Subject 009 had a baseline hemoglobin value of 9.8 g/dL (protocol limit was 10 g/dL). Subject 013 had a baseline hemoglobin value of 11.1 g/dL (lower limit of reference range=11.6 g/dL). Subject 014 had baseline results for hemoglobin and hematocrit of 12.6 g/dL and 37.9%, respectively (lower limit of reference range=13.2 g/dL for hemoglobin and 38.5% for hematocrit).

Results of Pharmacokinetic and Statistical Analyses

Pharmacokinetics of Oxymorphone and Metabolites in Plasma

The average plasma concentrations are plotted in FIG. 11. As noted in FIG. 11, subjects with hepatic impairment had higher plasma concentrations of oxymorphone and 6-OH-oxymorphone than healthy controls (matched for age, gender, and weight). The average plasma concentrations of oxymorphone-3-glucuronide were similar in all three treatment groups. The average oxymorphone and 6-OH-oxymorphone concentrations in subjects with moderate-severe hepatic impairment significantly exceeded those of both the healthy controls and subjects with mild hepatic impairment. The relative difference between the treatment groups was higher for oxymorphone than for 6-OH-oxymorphone.

Mean (±SD) pharmacokinetic results for oxymorphone, 6-OH-oxymorphone, and oxymorphone-3-glucuronide are summarized by treatment group in the following table (Table 34).

TABLE 34
Mean (SD) Plasma Pharmacokinetic Results

| | | Moderate-Severe | |
|---|---|---|---|
| Analyte Variable | Mild Impairment | Impairment | Healthy Controls |
| Oxymorphone | | | |
| AUC (ng · hr/mL) | 32.10 (21.71) | 105.57 (108.92) | 20.58 (9.02) |
| AUCT (ng · hr/mL) | 29.96 (21.79) | 103.66 (108.86) | 17.96 (7.69) |
| Cₘₐₓ (ng/mL) | 3.98 (4.06) | 9.16 (9.76) | 1.73 (0.68) |
| Tₘₐₓ (hr)* | 1.75 (0.5-5.0) | 1.5 (0.5-5.0) | 3.5 (1.0-12.0) |
| λ_{z} (hr⁻¹) | 0.0830 (0.0392) | 0.0970 (0.0365) | 0.0970 (0.0540) |
| T½ (hr) | 11.82 (9.34) | 8.04 (2.90) | 9.98 (6.35) |
| CL/F (L/min) | 13.30 (5.81) | 7.17 (5.08) | 21.15 (13.54) |
| 6-OH-oxymorphone | | | |
| AUC (ng · hr/mL) | 17.35 (11.17) | 32.12 (19.80) | 14.76 (11.51) |
| AUCT (ng · hr/mL) | 14.11 (9.73) | 29.25 (20.17) | 10.80 (9.65) |
| Cₘₐₓ (ng/mL) | 1.12 (0.79) | 1.78 (1.00) | 0.72 (0.32) |
| Tₘₐₓ (hr)* | 1.25 (0.5-3.0) | 1.75 (1.0-6.0) | 1.25 (1.0-4.0) |
| λ_{z} (hr⁻¹) | 0.0545 (0.0269) | 0.0632 (0.0303) | 0.0532 (0.0356) |
| T½ (hr) | 17.54 (13.12) | 13.87 (8.02) | 19.19 (11.75) |
| Oxymorphone-3-glucuronide | | | |
| AUC (ng · hr/mL) | 2954.1 (1136.2) | 2713.9 (1498.6) | 2979.6 (896.1) |
| AUCT (ng · hr/mL) | 2836.7 (1091.9) | 2629.3 (1457.8) | 2886.8 (883.9) |
| Cₘₐₓ (ng/mL) | 274.1 (95.8) | 212.0 (100.5) | 234.3 (41.7) |
| Tₘₐₓ (hr)* | 3.0 (3.0-4.0) | 3.5 (3.0-4.0) | 3.0 (1.5-4.0) |
| λ_{z} (hr⁻¹) | 0.0974 (0.0367) | 0.1075 (0.0435) | 0.0859 (0.0264) |
| T½ (hr) | 8.21 (3.87) | 7.40 (2.98) | 8.89 (3.00) |

*median (range)

The most substantial pharmacokinetic differences between the treatment groups were observed for the parent compound (oxymorphone). The mean oxymorphone AUC ranged from 20.58 (9.02) in the control group to 32.10 (21.71) and 105.57 (108.92) ng·hr/mL in subjects with mild and moderate-severe hepatic impairment, respectively. The AUC results were significantly more variable in the groups with hepatic impairment than in the healthy control group. Individual oxymorphone AUC results ranged from 15.35 to 74.84 ng·hr/mL in subjects with mild impairment; from 24.85 to 250.32 ng·hr/mL in subjects with moderate-severe impairment; and from 6.18 to 33.85 ng·hr/mL in healthy controls. Mild hepatic impairment was associated with an increase in average oxymorphone AUC of approximately 1.56-fold relative to controls, while moderate-severe impairment was associated with an increase of 5.13-fold. The mean oxymorphone “Cₘₐₓ” ranged from 1.73 (0.68) ng/mL in the control group to 3.98 (4.06) ng/mL and 9.16 (9.76) ng/mL in subjects with mild and moderate-severe hepatic impairment, respectively. As with AUC, there was substantial variability in oxymorphone Cₘₐₓ among subjects with mild impairment (range 0.86 to 11.40 ng/mL) and moderate-severe impairment (range 1.66 to 23.59 ng/mL); Cₘₐₓ in the control group ranged from 0.64 to 2.83 ng/mL.

The mean plasma AUC results for 6-OH-oxymorphone were also higher in subjects with mild and moderate-severe hepatic impairment than in healthy controls. The relative increases in 6-OHoxymorphone AUC were substantially larger in subjects with moderate-severe impairment (2.18-fold over control) than in subjects with mild impairment (1.18-fold over control).

No large differences in oxymorphone-3-glucuronide pharmacokinetic results were observed. The elimination rates for oxymorphone and its metabolites did not appear to be significantly altered by hepatic impairment.

The pharmacokinetic results for oxymorphone, 6-OH-oxymorphone, and oxymorphone-3-glucuronide were compared between treatment groups following log-transformation (natural log). The results of this analysis are displayed in FIG. 12 (oxymorphone), 13 (6-OH-oxymorphone) and 14 (oxymorphone-3-glucuronide).

The log-transformed results for the AUC of oxymorphone show at the 90 percent confidence interval a ratio of about 0.9 to about 2.5 for AUC of the mildly impaired to AUC of a healthy patient, for example about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, or about 2.5.

The log-transformed results for the Cₘₐₓ of oxymorphone show at the 90 percent confidence interval a ratio of about 0.9 to about 2.7 for Cₘₐₓ of the mildly impaired to Cₘₐₓ of a healthy patient, for example about for example about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, or about 2.7.

The log-transformed results for the AUC of 6-OH-oxymorphone show at the 90 percent confidence interval a ratio of about 0.8 to about 2.3 for AUC of the mildly impaired to AUC of a healthy patient, for example about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, or about 2.3.

The log-transformed results for the Cₘₐₓ of 6-OH-oxymorphone show at the 90 percent confidence interval a ratio of about 0.9 to about 2.1 for Cₘₐₓ of the mildly impaired to Cₘₐₓ of a healthy patient, for example about for example about 0.9, about 1.0, about 1.1, about 1.2, about 1.3, about 1.4, about 1.5, about 1.6, about 1.7, about 1.8, about 1.9, about 2.0, or about 2.1.

Relative to healthy controls, oxymorphone AUC was increased 1.52-fold in subjects with mild hepatic impairment and 3.61-fold in subjects with moderate-to-severe hepatic impairment. The mean oxymorphone Cₘₐₓ was increased by 1.68- and 3.41-fold in subjects with mild and moderate-severe impairment, respectively. The results indicate that the relative bioavailability of oxymorphone is significantly increased in hepatic impairment. The amount of increase in oxymorphone bioavailability is substantially higher in subjects with moderate-severe impairment than in subjects with mild impairment.

Examination of individual AUC results indicate that the observed AUC values for three (3) of the six (6) subjects with mild hepatic impairment fall within the range observed in the healthy control group. Although the lower 90% confidence limits for AUC and Cₘₐₓ in the group with mild hepatic impairment overlap 1.0, the point estimates exceed the control group by 50% and individual results indicate that most of the overlap occurs in the upper range of values observed within the control group.

The oxymorphone elimination rate constant was slightly reduced in subjects with mild impairment (ratio=0.87, 90% confidence limits=0.58-1.31) and slightly increased in subjects with moderate-severe impairment (ratio=1.11, 90% confidence limits=0.74-1.66). It is not clear whether the results demonstrate a real difference from healthy controls, but the lack of consistency in the direction of change suggests that there are no clinically significant differences.

The 6-OH-oxymorphone AUC and Cₘₐₓ in subjects with moderate-severe hepatic impairment was increased 2.35-fold relative to the control group. In subjects with mild hepatic impairment the AUC and Cₘₐₓ were increased by 1.36- and 1.40-fold, respectively. Examination of individual AUC and Cₘₐₓ results for subjects with mild hepatic impairment indicates that there is substantial overlap with the upper half of the control group.

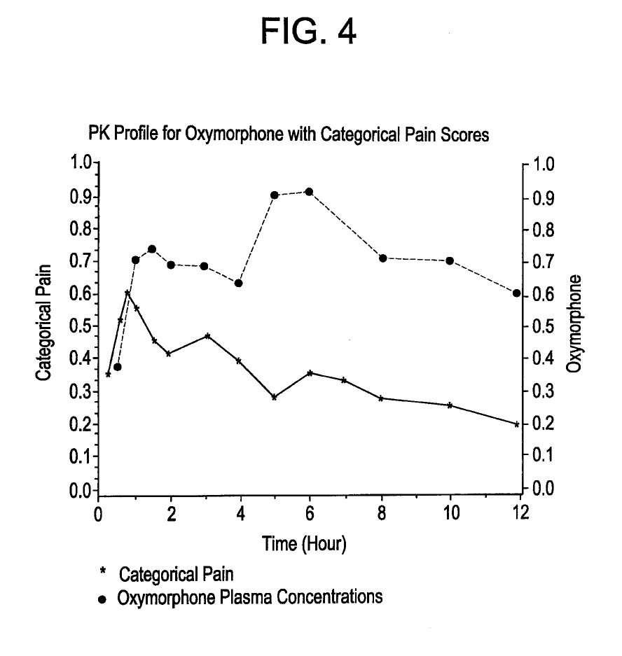

The results for oxymorphone-3-glucuronide (FIG. 4) AUC and “Cₘₐₓ” indicate that there is very little difference between subjects with mild hepatic impairment and healthy controls; the point estimates were 0.972 and 1.133 for AUC and Cₘₐₓ, respectively. The AUC and Cₘₐₓ results in subjects with moderate-severe impairment were approximately 80% of control.

In order to further examine the metabolism of oxymorphone in subjects with hepatic impairment, the AUC values were converted to molar concentrations and the ratios of metabolite-to-parent were calculated. The results are summarized in FIG. 15.

The ratio of 6-OH-oxymorphone:oxymorphone and oxymorphone-3-glucuronide:oxymorphone decreased in association with the degree of hepatic impairment. The mean (±SD) ratios for 6-OH-oxymorphone:oxymorphone were 0.66 (0.30), 0.55 (0.10), and 0.49 (0.37) in controls and subjects with mild and moderate-severe impairment, respectively. Although the AUC for 6-OH-oxymorphone appears to be increased in subjects with hepatic impairment, the metabolite ratios suggest that liver disease is associated with a small reduction the conversion of oxymorphone to 6-OH-oxymorphone. Based on mean results, the conversion of oxymorphone to 6-OH-oxymorphone appears to be reduced by approximately 17% and 26% in subjects with mild and moderate-severe hepatic impairment, respectively.

The mean (±SD) ratios for oxymorphone-3-glucuronide:oxymorphone were 104.1 (39.5), 67.2 (27.0), and 45.52 (39.2) in controls and subjects with mild and moderate-severe impairment, respectively. While the plasma AUC's for oxymorphone-3-glucuronide did not significantly differ between the three treatment groups, circulating amounts of the 3-glucuronide were substantially reduced relative to the parent compound. The circulating concentrations of oxymorphone-3-glucuronide exceed those of the parent compound by approximately 100-fold in the healthy control group, but by only 45-fold in subjects with moderate-severe impairment. The mean metabolite ratios suggest that conversion of oxymorphone to oxymorphone-3-glucuronide is reduced by approximately 35% and 56% in subjects with mild and moderate-severe hepatic impairment, respectively.

Pharmacokinetics of Oxymorphone and Metabolites in Urine

The concentration of oxymorphone, 6-OH-oxymorphone, and oxymorphone-3-glucuronide was measured in urine samples collected from the time of dose administration through 120 hours after dose administration. In addition to the direct metabolite assays, the concentrations of oxymorphone and 6-O-Hoxymorphone were measured in urine samples after hydrolysis with β-glucuronidase. The results obtained prior to hydrolysis were subtracted from the results obtained after hydrolysis to provide an indirect measure of oxymorphone and 6-OH-oxymorphone conjugates. Examination of these results indicates that oxymorphone-3-glucuronide appears to account for virtually all of the oxymorphone conjugates. The results further indicate that glucuronide conjugates of the 6-OH metabolite are present in the urine. The position of conjugation cannot be determined from this assay, but it appears that conjugates of 6-OH-oxymorphone are present in higher amounts than the unconjugated form of the metabolite.

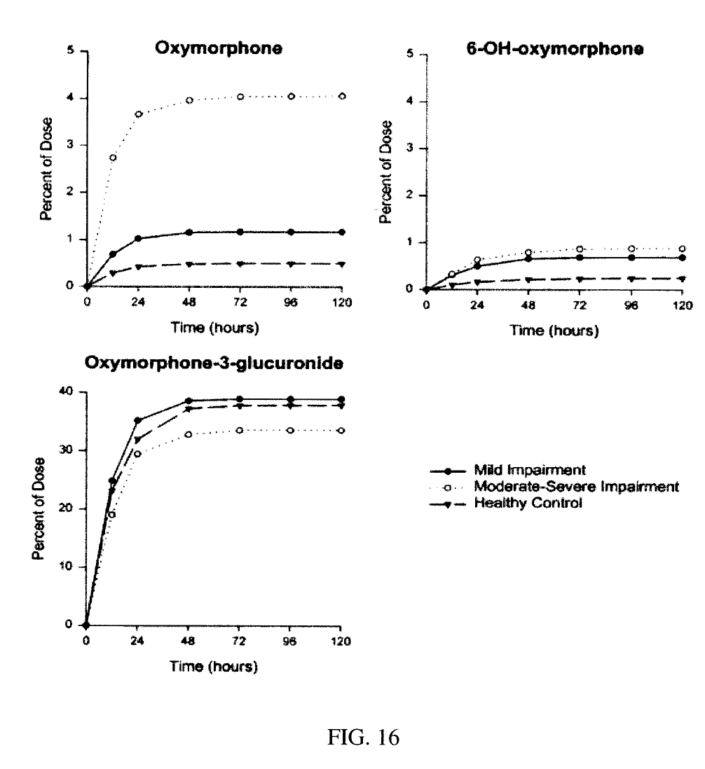

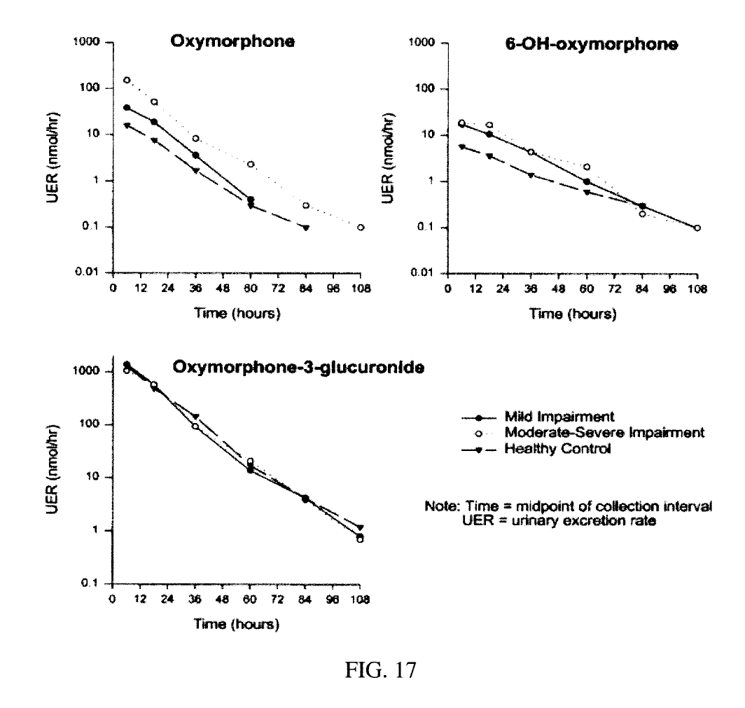

The cumulative amount of oxymorphone, 6-OH-oxymorphone, and oxymorphone-3-glucuronide excreted in the urine is displayed in the graphs of FIG. 16.

Moderate-severe hepatic impairment was associated with an approximately 8-fold increase in the amount of oxymorphone excreted in the urine. The mean amount of oxymorphone excreted unchanged in the urine was 0.5%, 1.2%, and 4.1% in the control group and in subjects with mild or moderate-severe hepatic impairment, respectively. Consistent with the plasma AUC results, subjects with hepatic impairment excreted a higher amount of 6-OHoxymorphone in the urine than the controls. The mean amount of 6-OH-oxymorphone excreted in the urine was 0.25%, 0.71%, and 0.88% in the control group and in subjects with mild or moderate-severe hepatic impairment, respectively. The mean amount of oxymorphone-3-glucuronide excreted in the urine was 38.1%, 39.3%, and 33.7% in the control group and in subjects with mild or moderate-severe hepatic impairment, respectively.

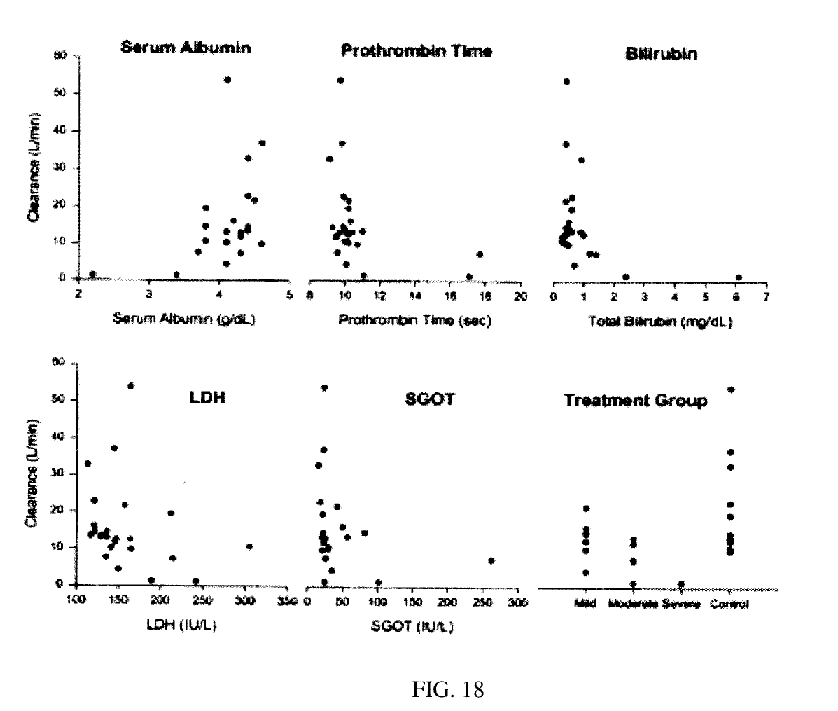

The ratio of metabolite-to-parent excreted in the urine followed a pattern very similar to that seen in the plasma. Although the percent of administered dose excreted in the urine as oxymorphone-3-glucuronide is similar in the three treatment groups, the amount of metabolite excreted relative to the amount of oxymorphone excreted unchanged was substantially different. The mean ratio of oxymorphone-3-glucuronide:oxymorphone excreted in the urine was 79.05, 39.43, and 33.76 in healthy controls and subjects with mild or moderate-severe hepatic impairment, respectively. If the route of excretion for the metabolite is unchanged, the results indicate that conversion of oxymorphone to oxymorphone-3-glucuronide is reduced by approximately 50% and 57% in mild and moderate-severe hepatic impairment, respectively. The similarity in urinary excretion rates between the three groups (FIG. 17), suggests that renal excretion is unaffected by liver disease.

Urinary excretion of unchanged oxymorphone was essentially complete by the end of the 48 to 72 hour collection interval; urinary excretion of 6-OH-oxymorphone was essentially complete by 96 hours; and only small amounts of oxymorphone-3-glucuronide were excreted during the 96 to 120 hour collection interval.

Urinary excretion rate constants for oxymorphone and its metabolites were slightly higher in subjects with hepatic impairment than in controls, but there were no clinically meaningful differences in urinary excretion rates. As suggested in FIG. 17 (i.e., the urinary excretion rate plots are essentially parallel), these results for mean urinary excretion rate constants indicate that urinary excretion is not impaired in this population with hepatic disease. This data supports a conclusion that the metabolic ratios are reflecting differences in the degree of impairment in oxymorphone metabolism rather than differential excretion rates.

Relationship Between Oxymorphone Oral Clearance and Measures of Hepatic Function

The potential relationship between oxymorphone clearance and potential indicators of hepatic impairment was explored by the use of scatter plots (FIG. 18) and tests of association.

As reflected in the prior pharmacokinetic analyses, the oral clearance of oxymorphone differed significantly between the three treatment groups (p=0.0112). A nonparametric correlation analysis indicated that there were statistically significant correlations between oxymorphone oral clearance and prothrombin time, serum albumin, SGOT, and LDH. However, as indicated in FIG. 18, none of these relationships were particularly strong.

The hepatically impaired subjects with the most extreme oxymorphone pharmacokinetic results are summarized in the following table (Table 35).

TABLE 35
Characteristics of Subjects with Very Low Oxymorphone Clearance Values

| | CL/F | AUC | Cmax | Albumin | SGOT | LDH | Bilirubin | PT |
|---|---|---|---|---|---|---|---|---|
| Subject | (L/min) | (ng · hr/mL) | (ng/mL) | (g/dL) | (IU/L) | (IU/L) | (mg/dL) | (sec) |
| 006 (B)* | 7.34 | 45.40 | 4.31 | 4.3 | 262 | 215 | 1.4 | 17.7 |
| 007 (A)* | 4.45 | 74.84 | 11.40 | 4.1 | 35 | 150 | 0.7 | 10.1 |
| 009 (B)* | 1.38 | 241.14 | 19.57 | 3.4 | 25 | 189 | 2.4 | 11.1 |
| 010 (B)* | 7.63 | 43.67 | 3.72 | 3.7 | 26 | 135 | 1.2 | 9.6 |
| 011 (C)* | 1.33 | 250.32 | 23.59 | 2.2 | 102 | 242 | 6.1 | 17.1 |

*Child-Pugh Class
PT = prothrombin time

Mean oxymorphone oral clearance in the control group was 21.15 L/min (range 9.85 to 53.97 L/min). Five (5) subjects with hepatic impairment (Subjects 006, 007, 009, 010, and 011) had very low oral clearance values which were well below the lower end of the healthy control group; and also had significantly elevated oxymorphone AUC and Cₘₐₓ values (Table 35). One of these subjects was in Child-Pugh Class A (Subject 007), three were in Child-Pugh Class B (Subjects 006, 009, and 010), and one was in Child-Pugh Class C (Subject 011). While some general correlation was present for SGOT and LDH, elevations in hepatic enzymes does not appear to be a good predictor of substantial changes in oxymorphone clearance since most of these subjects had results within the normal range. The two subjects with the lowest oxymorphone clearances (Subjects 009 and 011) both had low serum albumin concentrations (3.4 and 2.2 g/dL, respectively), elevated serum bilirubin levels (2.4 and 6.1 mg/dL, respectively), and prothrombin times above the upper limit of normal (11.1 and 17.1 sec, respectively).

Discussion

Following oral administration, oxymorphone is subjected to extensive first-pass metabolism, is highly metabolized, and can be categorized as a highly extracted drug. Following administration of an oral solution, oxymorphone has been shown to have an average absolute bioavailability of approximately 10%. Less than 1% of the administered dose is excreted unchanged in the urine. Oxymorphone bioavailability following administration of EN3202 tablets is equivalent to an oral solution; indicating that slowing the rate of oxymorphone delivery reduces the peak plasma concentrations but does not change the absolute bioavailability. Due to the fact that oxymorphone is a highly extracted drug and subject to a high degree of first pass metabolism, a significant increase in oxymorphone bioavailability can be expected in patients with impaired hepatic function.

Within the current trial, the mean (±SD) oxymorphone AUC and Cₘₐₓ values in healthy control subjects were 20.58 (9.02) ng·hr/mL and 1.73 (0.68) ng/mL, respectively. The corresponding values for AUC and Cₘₐₓ in subjects with hepatic disease were 32.10 (21.71) ng·hr/mL and 3.98 (4.06) ng/mL in subjects with mild disease, and 105.57 (108.92) ng·hr/mL and 9.16 (9.76) ng/mL in subjects with moderate or severe disease. Individual oxymorphone AUC values varied across a 5-fold range (6.2 to 33.9 ng·hr/mL) in healthy controls, a 5-fold range (15.4 to 74.8 ng·hr/mL) in subjects with mild disease, and a 10-fold range (24.9 to 250.3 ng·hr/mL) in subjects with moderate-severe disease.

There was not much difference in the median oxymorphone AUC between the mildly-impaired group (24.7 ng·hr/mL) and the control group (23.8 ng/mL). Only one of six subjects in this group (Subject 007, AUC=74.84 ng·hr/mL) had an oxymorphone AUC value outside the range observed in healthy subjects. As a result, the relative difference in mean AUC between the mild group and the control group (mean ratio=1.52) is substantially due to one subject and probably overestimates the effect for the majority of subjects with mild liver disease. The mean oxymorphone AUC for the mild group, after excluding Subject 007, is only 23.55 ng·hr/mL.

The mean bioavailability of oxymorphone was increased 3,6-fold (90% confidence interval 2.4 to 6.6) in subjects with moderate or severe liver disease relative to healthy controls. Two subjects in this group had very high oxymorphone plasma levels relative to controls; one was Child-Pugh Class C (Subject 011, AUC=250.32 ng·hr/mL and Cₘₐₓ=23.59 ng/mL) and the other was Child-Pugh Class B (Subject 009, AUC=241.14 ng·hr/mL and Cₘₐₓ=19.57 ng/mL). Two of the remaining subjects with moderate hepatic impairment had AUC values, which were essentially twice the mean of the control group, and the other two subjects in the group had AUC values, which were within the upper range of control.

As reported in a previous study, the average absolute bioavailability of orally administered oxymorphone is approximately 10%. As a result, complete elimination of first-pass metabolism (i.e., increasing absolute bioavailability from 10% to 100%) could be expected to result in a 10-fold increase in oxymorphone AUC. Changes of this magnitude have been observed in one subject with moderate and one subject with severe liver disease. While the oral bioavailability of oxymorphone was clearly increased in subjects with moderate or severe liver disease, this change was not associated with increases in the plasma elimination half-life for oxymorphone, 6-OH-oxymorphone, or oxymorphone-3-glucuronide. Even in association with apparent 10-fold increases in oxymorphone bioavailability, the plasma oxymorphone elimination half-life was 6.96 hours and 10.67 hours, in Subjects 009 and 011, respectively. Considering these factors, it appears reasonable to conclude that results at or near the maximal level of oxymorphone bioavailability have been observed in the present study.

The mean plasma AUC and Cₘₐₓ for 6-OH-oxymorphone was increased approximately 2,3-fold relative to the healthy control group in subjects with moderate or severe hepatic impairment. Mean plasma concentrations of oxymorphone-3-glucuronide did not differ significantly between the three treatment groups. However, plasma and urine metabolite ratios indicate that the conversion of oxymorphone to oxymorphone-3-glucuronide is significantly impaired in the group with moderate-severe liver disease. The mean metabolite-to-parent ratios for oxymorphone-3-glucuronide, in both plasma and urine, were 2,3-fold lower in subjects with moderate-severe impairment than in the control group. Conversion of oxymorphone to 6-OH-oxymorphone appears to be reduced in subjects with moderate-severe impairment, but to a lesser extent than conversion to the 3-glucuronide.

While there was no single good predictor of oxymorphone clearance, the two subjects with the lowest oxymorphone clearances (Subjects 009 and 011) both had low serum albumin concentrations (3.4 and 2.2 g/dL, respectively), elevated serum bilirubin levels (2.4 and 6.1 mg/dL, respectively), and prothrombin times above the upper limit of normal (11.1 and 17.1 sec, respectively).

The results of this study demonstrate that moderate or severe hepatic impairment (Child-Pugh Class B or C) is associated with a significant increase in oxymorphone bioavailability and a reduction in the conversion of oxymorphone to both 6-OH-oxymorphone and oxymorphone-3-glucuronide. Subjects with moderate or severe liver disease may have clinically significant increases in plasma oxymorphone concentrations; mean oxymorphone AUC was increased 3,6-fold for the group as a whole, and up to 10-fold in two of six individual subjects. The majority of subjects with mild liver disease (Child-Pugh Class A) do not appear to have a significant increase in oxymorphone bioavailability relative to healthy controls. Since oxymorphone is subject to high first-pass metabolism and these results have confirmed increased bioavailability, particularly in moderate-severe hepatically impaired patients, caution should be exercised in initial dosing and slow titration of these patients.

All references, including publications, patent applications, and patents, cited herein are hereby incorporated by reference to the same extent as if each reference there individually and specifically indicated to be incorporated by reference were set forth in its entirety herein.

The use of the terms “a” and “an” and “the” and similar referents in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the invention.

Alternative embodiments of the claimed invention are described herein, including the best mode known to the inventors for carrying out the claimed invention. Of these, variations of the disclosed embodiments will become apparent to those of ordinary skill in the art upon reading the foregoing disclosure. The inventors expect skilled artisans to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the claimed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value. Thus, as a general matter, “about” or “approximately” broaden the numerical value. Also, the disclosure of ranges is intended as a continuous range including every value between the minimum and maximum values plus the broadening of the range afforded by the use of the term “about” or “approximately”. Thus, recitation of ranges of values herein are merely intended to serve as a shorthand method of referring individually to each separate value falling within the range, unless otherwise indicated herein, and each separate value is incorporated into the specification as if it were individually recited herein.

1-20. (canceled)

21. A method of providing extended pain relief to patients in need thereof comprising:
informing the patients or the patients' prescribing physicians that the average bioavailability of oxymorphone in an oral extended release dosage form designed to have a 12 hour dosing cycle is increased by at least about 360% for subjects with moderate hepatic impairment compared to that for healthy subjects,
providing a therapeutically effective amount of such an extended release oral dosage form containing between about 5 mg and about 40 mg of oxymorphone or a pharmaceutically acceptable salt thereof; and
orally administering the dosage form to the patients, wherein upon placement of the dosage form in an in vitro dissolution test comprising USP Paddle Method at 50 rpm in 500 ml media having a pH of 1.2 to 6.8 at 37° C., releases about 15% to about 50%, by weight, of the oxymorphone or salt at about 1 hour into the test, releases about 45% to about 80%, by weight, of the oxymorphone or salt thereof at about 4 hours into the test, and releases at least about 80%, by weight, of the oxymorphone or salt thereof at about 10 hours into the test.

22. The method of claim 21 wherein in the increase in average oral bioavailability of at least about 152% is associated with mild hepatic impairment, and an increase in average oral bioavailability of about 1220% is associated with severe hepatic impairment, in each case in comparison to that for healthy subjects.

23. The method of claim 21 wherein the information is provided at least via a label associated with the extended release oral dosage form of oxymorphone.

24. The method of claim 21 wherein the information further contains a recommendation that subjects with hepatic impairment be initially administered the lowest available dose of the extended release oral dosage form of oxymorphone or its salt.

25. The method of claim 21 wherein the information further indicates that the half-life of the oxymorphone is not significantly affected by hepatic impairment.

26-31. (canceled)

32. A method of providing extended pain relief to patients in need thereof comprising:
packaging an oral extended release formulation containing between about 5 mg and about 40 mg of oxymorphone or its pharmaceutically acceptable salt with directions that it be administered every 12 hours and with information that the average bioavailability of oxymorphone in an extended release formulation designed to have a 12 hour dosing cycle is increased by at least about 360% for subjects with moderate hepatic impairment compared to that for healthy subjects; and
orally administering to the patients a therapeutically effective amount of said packaged extended release formulation of oxymorphone or a pharmaceutically acceptable salt thereof, wherein upon placement of the extended release formulation in an in vitro dissolution test comprising USP Paddle Method at 50 rpm in 500 ml media having a pH of 1.2 to 6.8 at 37° C, releases about 15% to about 50%, by weight, of the oxymorphone or salt at about 1 hour into the test, releases about 45% to about 80%, by weight, of the oxymorphone or salt thereof at about 4 hours into the test, and releases at least about 80%, by weight, of the oxymorphone or salt thereof at about 10 hours into the test.

33. The method of claim 32 wherein in the information an increase in average oral bioavailability of at least about 152% is associated with mild hepatic impairment, and an increase in average oral bioavailability of about 1220% is associated with severe hepatic impairment, in each case in comparison to that for healthy subjects.

34. The method of claim 32 wherein the packaged information is provided at least via a label associated with the packaged extended release formulation of oxymorphone.

35. The method of claim 32 wherein the directions further contain a recommendation that patients with hepatic impairment be initially administered the lowest available dose of the packaged extended release formulation of oxymorphone or its salt.

36. The method of claim 32 wherein the packaged information further indicates that the half-life of the oxymorphone is not significantly affected by hepatic impairment.

37. The method of claim 32, wherein the ratio of the log transformed plasma AUC of oxymorphone of an impaired patient to that of a healthy patient is about 0.9 to about 2.5 if both patients were administered the same dose of the formulation.

38. The method of claim 32, wherein the ratio of the log transformed plasma Cₘₐₓ of oxymorphone of an impaired patient to that of a healthy patient is about 0.9 to about 2.7 if both patients were to be administered the same dose of the formulation.

39. The method of claim 32, wherein the ratio of the log transformed plasma AUC of 6-OH-oxymorphone of an impaired patient to that of a healthy patient is about 0.8 to about 2.3 if both patients were administered the same dose of the formulation.

40. The method of claim 32, wherein the ratio of the log transformed plasma Cₘₐₓ of 6-OH-oxymorphone of an impaired patient to that of a healthy patient is about 0.9 to about 2.1 if both patients were to be administered the same dose of the formulation.

41. A method of using oxymorphone in the treatment of pain in a patient having mild hepatic impairment in need thereof, comprising:
providing a patient having mild hepatic impairment with a therapeutically effective amount of a controlled release oral dosage form containing about 5 mg to about 40 mg of oxymorphone or a pharmaceutically acceptable salt thereof;
informing the patient or the patient's prescribing physician that the bioavailability of oxymorphone may be increased in patients with hepatic impairment; and
orally administering the dosage form of oxymorphone to the patient,
wherein the ratio of the log transformed plasma AUC of oxymorphone of an impaired patient to that of a healthy patient is about 0.9 to about 2.5 if both patients were administered the same dose of the oxymorphone or salt thereof.
